SEQUENCE LISTING (6 sequences)

SEQ ID NO: 1 - DNA, 21 bp, Artificial Sequence
  synthetic oligonucleotide
agccatgaacggcacagaggg

SEQ ID NO: 2 - DNA, 22 bp, Artificial Sequence
  synthetic oligonucleotide
cttaggctggagccacctggct

SEQ ID NO: 3 - DNA, 37 bp, Artificial Sequence
  synthetic oligonucleotide
atgcgggccgccaccatggtgagcaagggcgaggagc

SEQ ID NO: 4 - DNA, 43 bp, Artificial Sequence
  synthetic oligonucleotide
aggtcttctcggacggcatcttgtacagctcgtccatgccgag

SEQ ID NO: 5 - DNA, 25 bp, Artificial Sequence
  synthetic oligonucleotide
atgccgtccgagaagaccttcaagc

SEQ ID NO: 6 - DNA, 34 bp, Artificial Sequence
  synthetic oligonucleotide
atctcgagttacacagccattgctgtcccgaatg

(19) United States
(12) Patent Application Publication
(10) Pub. No.: US 2011/0318424 A1
(43) Pub. Date: Dec. 29, 2011

(54) METHODS FOR PROLONGING VIABILITY OF CONE CELLS USING MODULATORS OF THE MAMMALIAN TARGET OF RAPAMYCINE (mTOR)

(75) Inventors: Constance Louise Cepko, Newton, MA (US); Claudio Punzo, Brookline, MA (US)

(73) Assignee: PRESIDENT AND FELLOWS OF HARVARD COLLEGE, Cambridge, MA (US)

(21) Appl. No.: 13/132,730

(22) PCT Filed: Dec. 3, 2009
(86) PCT No.: PCT/US09/66557

§ 371 Date: Sep. 14, 2011

Related U.S. Application Data
(60) Provisional application No. 61/119,689, filed on Dec. 3, 2008.
(60) Provisional application No. 61/120,122, filed on Dec. 5, 2008.

(51) Int. Cl. A61K 38/28; A61K 38/18; A61P 27/02; A61K 35/16; A61K 31/661; A61K 31/198; C12N 5/071; A61K 38/30

(52) U.S. Cl. 424/531; 435/375; 514/5.9; 514/7.6; 514/8.6; 514/8.5; 514/129; 514/561

(57) ABSTRACT

The present invention is directed to the use of modulators of the mammalian target of rapamycine (mTOR) pathway, glucose and/or glucose enhancers for treating retinal disorders and, in particular, for prolonging the viability of cone cells.

### RELATED APPLICATIONS

This application claims the benefit of and priority to U.S. Provisional Patent Application Ser. No. 61/119,689, filed on Dec. 3, 2008, and U.S. Provisional Application Ser. No. 61/120,122, filed on Dec. 5, 2008. This application is also related to U.S. Provisional Patent Application Ser. No. 61/169,835, filed on Apr. 16, 2009. The entire contents of each of the foregoing provisional applications are incorporated herein by reference.

### STATEMENT REGARDING FEDERALLY SPONSORED RESEARCH OR DEVELOPMENT

This invention was made with Government support under contract EY014466 awarded by the National Institutes of Health. The government has certain rights in the invention.

### FIELD OF THE INVENTION

The present invention is directed to the use of modulators of the mammalian target of rapamycine (mTOR) pathway, glucose and/or glucose enhancers for prolonging the viability of cone cells.

### BACKGROUND OF THE INVENTION

The retina contains two major types of light-sensitive photoreceptor cells, i.e., rod cells and cone cells. Cone cells are responsible for color vision and require brighter light to function, as compared to rod cells. There are three types of cones, maximally sensitive to long-wavelength, medium-wavelength, and short-wavelength light (often referred to as red, green, and blue, respectively, though the sensitivity peaks are not actually at these colors). Cones are mostly concentrated in and near the fovea. Only a few are present at the sides of the retina. Objects are seen most sharply in focus when their images fall on this spot, as when one looks at an object directly. Cone cells and rods are connected through intermediate cells in the retina to nerve fibers of the optic nerve. When rods and cones are stimulated by light, the nerves send off impulses through these fibers to the brain.

Reduced viability of cone cells is associated with various retinal disorders, in particular, retinitis pigmentosa. Retinitis pigmentosa is a family of inherited retinal degenerations (RD) that is currently untreatable and frequently leads to blindness. Affecting roughly 1 in 3,000 individuals, it is the most prevalent form of RD caused by a single disease allele (RetNet, www.sph.uth.tmc.edu/Retnet/). The phenotype is characterized by an initial loss of night vision due to the malfunction and death of rod PRs, followed by a progressive loss of cones (Madreperla, S. A., et al. (1990) Arch Ophthalmol 108, 358-61). Additionally, retinitis pigmentosa is further characterised by the following manifestations: night blindness, progressive loss of peripheral vision, eventually leading to total blindness; ophthalmoscopic changes consist in dark mosaic-like retinal pigmentation, attenuation of the retinal vessels, waxy pallor of the optic disc, and in the advanced forms, macular degeneration. Since cones are responsible for color and high acuity vision, it is their loss that leads to a reduction in the quality of life. In many cases, the disease-causing allele is expressed exclusively in rods; nonetheless, cones die too. Indeed, to date there is no known form of RD in humans or mice where rods die, and cones survive. In contrast, mutations in cone-specific genes result only in cone death.

### SUMMARY OF THE INVENTION

The present invention is directed to the use of modulators of the mammalian target of rapamycine (mTOR) pathway for treating retinal disorders and, in particular, for prolonging the viability of cone cells. The present invention is based, at least in part, on the discovery that a modulator of the mTOR pathway can be used to prolong the viability of cone cells by decreasing and/or delaying cone cell death. Accordingly, the present invention provides methods for treating or preventing retinal disorders, in particular retinitis pigmentosa, and for prolonging the viability of cone cells, by contacting cone cells with an mTOR modulator.

In one aspect, the present invention is directed to a method for treating or preventing a retinal disorder in a subject by administering to the subject an mTOR modulator in an amount effective for modulating mTOR activity in the subject, thereby treating or preventing the retinal disorder. In a particular embodiment, the retinal disorder is retinitis pigmentosa. In various embodiments, the retinal disorder is associated with decreased viability of cone and/or rod cells. In another embodiment, the retinal disorder is a genetic disorder. In yet another embodiment, the retinal disorder is not diabetic retinopathy. Alternatively or in addition, the retinal disorder is not associated with blood vessel leakage and/or growth.

In another aspect, the present invention is directed to a method for treating or preventing retinitis pigmentosa in a subject by administering to the subject an mTOR modulator in an amount effective for modulating mTOR activity in the subject, thereby treating or preventing retinitis pigmentosa.

In yet another aspect, the present invention is directed to a method for prolonging the viability of a cone cell, by contacting the cone cell with an mTOR modulator in an amount effective for modulating mTOR activity in the cell, thereby prolonging the viability of the cone cell, e.g., for about 1 week, about 2 weeks, about 3 weeks, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, about 12 months, about 2 years, about 3 years, about 4 years, about 5 years, about 10 years, about 15, years, about 20 years, about 25 years, about 30 years, about 40 years, about 50 years, about 60 years, about 70 years, and about 80 years.

In certain embodiments of any of the preceding aspects of the invention, the mTOR modulator is selected from the group consisting of insulin, growth factors, IGF-1, IGF-2, mitogens, serum, phosphatidic acid, amino acids, leucine, and analogues or derivatives thereof. In a particular embodiment, the mTOR modulator is insulin. Alternatively, in another embodiment, the mTOR modulator is not insulin. In other embodiments, the mTOR modulator stimulates mTOR phosphorylation. Alternatively, or in addition, the mTOR modulator activates a receptor and/or a signal transduction cascade upstream of mTOR. In one embodiment, the mTOR modulator is a glucose enhancer.

In another aspect, the present invention is directed to a method for treating or preventing a retinal disorder, for example, retinitis pigmentosa, in a subject by enhancing the intracellular levels of glucose in the subject, thereby treating or preventing the retinal disorder. In certain embodiments, the retinal disorder is associated with decreased viability of cone and/or rod cells. In other embodiments, the retinal disorder is a genetic disorder. In another embodiment the retinal disorder is not diabetic retinopathy. In yet another embodiment, the retinal disorder is not associated with blood vessel leakage and/or growth.

In another aspect, the present invention provides methods for treating or preventing retinitis pigmentosa in a subject by enhancing the intracellular levels of glucose in the subject, thereby treating or preventing retinitis pigmentosa.

In various embodiments of these aspects of the invention, the subject may be administered glucose in an amount effective to enhance the intracellular levels of glucose in the subject. For example, the glucose may be administered to the subject intravenously. In another embodiment, the subject may be administered a composition comprising a glucose enhancer in an amount effective to enhance the intracellular levels of glucose in the subject. In one embodiment, the glucose enhancer modulates biochemical pathways leading to enhanced intracellular glucose levels. In a particular embodiment, the glucose enhancer can serve to increase uptake of glucose into cells, for example rod and/or cone cells.

In another aspect, the present invention provides methods for prolonging the viability of a cone cell by enhancing the intracellular levels of glucose in the cell, thereby prolonging the viability of the cone cell, e.g., for about 1 week, about 2 weeks, about 3 weeks, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, about 12 months, about 2 years, about 3 years, about 4 years, about 5 years, about 10 years, about 15, years, about 20 years, about 25 years, about 30 years, about 40 years, about 50 years, about 60 years, about 70 years, and about 80 years. In a particular embodiment, the cone cell is exposed to glucose in an amount effective to enhance the intracellular levels of glucose in the cone cell. Alternatively, or in addition, the cone cell is exposed to a glucose enhancer in an amount effective to enhance the intracellular levels of glucose in the cone cell. For example, the glucose enhancer may serve to modulate biochemical pathways leading to enhanced intracellular glucose levels. In a particular embodiment, the glucose enhancer can serve to increase uptake of glucose into cells, for example rod and/ or cone cells.

Other features and advantages of the invention will be apparent from the following detailed description and claims

### BRIEF DESCRIPTION OF THE DRAWINGS

FIGS. 1a-1q depict rod death kinetics in the Rho-KO mutant described in Example 1 as follows: (a-d) Onset of rod death seen by cleaved nuclear envelope protein LaminA (a), Cleaved Caspase3 (b) (arrowheads) as well as TUNEL (c, d) (arrows) (dark gray in a, b shows nuclear DAPI staining). (d) Shows a retinal flat mount with view onto the photoreceptor layer. (e-h) Progression of rod death determined by the reduction of the ONL as seen by HE staining. (i-q) End phase of rod death assessed by section analysis (i-l) or by retinal flat mounts (m-q). In the Rho-KO the onset of rod death is around PW5 (a) and progresses up to PW25 (l). By PW17 the ONL is reduced to one row of cells (h, j) and in the following 8 weeks the remaining rods die (j-q) as seen by immunofluorescence with an antibody directed against guanine nucleotide protein alpha transducin (GnatI) on sections of progressively older animals (j-l). (m-q) Retinal flat mounts showing rods visualized by immunofluorescence with an antibody directed against GnatI. (m) Shows entire retina while (n, o) show higher magnification around the optic nerve head and (p) shows peripheral region. (q) Shows no signal at PW25 where on sections rods were also not detected (l). Age (in postnatal weeks (PW)) is indicated in the panels. Vertical bar in (a-c, e-l) indicates thickness of the ONL.

FIGS. 2a-2q depict rod death kinetics in the PDE-γ-KO described in Example 1 as follows: (a-d) Onset of rod death seen by cleaved caspase 3 (a, b). At P12, misplaced and excess cells in the INL were dying as part of developmental cell death, as seen in a wild-type control (a) (arrowheads) while in the mutant, cells started to die in the ONL, where photoreceptors reside (b) (arrows). The onset of rod death was also seen by immunofluorescence for the cleaved nuclear envelope protein, LaminA (c) (arrows) as well as TUNEL (d) (arrows; dark gray in a-d shows nuclear DAPI staining). Progression of rod death was determined by the reduction of the thickness of the ONL, as seen by HE staining (e-h). (i-q) End phase of rod death was assessed by analysis of sections of progressively older animals. (i-m) Rods were visualized by immunofluorescence with α-rhodopsin or by in situ hybridization for rhodopsin (n-q). (i, j) Retinal section at P16 showing peripheral to central region. (i) Same picture as in (j) with nuclear DAPI stain. (k, l) Higher magnification of section in (i) showing peripheral (k) and central (l) region. As rods die in a central to peripheral manner, more rods were present in the periphery than in the center. By P20, the ONL was reduced to 1 row of cells and rods were found mainly in the periphery (compare arrow (periphery) in (n) to arrowhead (central). The remaining rods in the PDE-γ-KO died over 4 weeks (n-q) as seen on sections. By P49 (q) all rods had died in this mutant (o-q: periphery). Age (in postnatal days (P)) is indicated in the panels. Vertical bar in (a-h, k, l) indicates thickness of the ONL. Data for PDE-β−/− are not shown as they are comparable to the PDE-γ-KO and we have presented data on the rod death kinetics of this mutant in an earlier publication (IOVS, 2007, 48 (2): 849-857).

FIGS. 3a-3o depict cone death kinetics described in Example 1 as follows: (a) qRT-PCR analysis for Opnlsw during cone degeneration. Changes are in indicated as the logarithm of the relative concentration over time on the Y-axis while X-axis indicates postnatal weeks. (b-h, j, k, m-o) Show retinal flat mounts. (k) Shows a retinal section. Light gray signal shows PNA expression, dark gray signal shows red/green opsin expression (b, j-n) or blue opsin expression (c, d, o). (b-d) Wild type retina at P35. Red/green opsin (b) and PNA (c, d) expression were detected dorsal and ventral while blue opsin (c, d) was detected only ventrally. (e-g, j-o) Analysis in the PDE-β mutant. (e-g) Central to peripheral gradient of PNA and shortening of cone outer segments (OS). At P20, prior to the major cone death phase, there were fewer elongated OS in the center (e) as compared to the periphery. (f) High magnification of a central or peripheral (g) OS from (e). (h) Wild type OS (white line in f-h marks the OS). (i) Quantification of OS length in central and peripheral regions. The data represents an average of 15 measurements on 3 different retinae of 3 week old mice. With the shortening of OSs during degeneration, red/green opsin was localized throughout the membrane of the cell body and PNA, which detects an extracellular protein(s), was reduced to a small dot attached to the residual OS (j) (arrow: yellow shows red/green and PNA overlap). (k) High magnification of a cone showing red/green localization at the membrane of the main cell body (arrow). (l) Cross section showing red/green in cell body (arrows; j-l P70). Red/green opsin was detected mainly dorsal (l) during degeneration while PNA (m, n) or blue opsin (o) were not altered (m, n: P21, same scale bar; o: P49).

FIGS. 4a-4g depict rod death kinetics in the P23H mutant described in Example 1 as follows. (a-c) Onset of rod death. As rod death progressed very slowly in this mutant, the upregulation of glial fibrillary acidic protein (GFAP) in Muller glia, which has been described as a hallmark of retinal degeneration, was used in conjunction with the other markers to determine the onset of rod degeneration. As seen by antibody staining against GFAP (a, b) degeneration started around PW10 (b). At PW5, GFAP was only found in the ganglion cell layer where it is normally expressed in astrocytes. Consistent with the upregulation of GFAP at PW10, cells positive for cleaved nuclear envelope protein LaminA (c) were also detected (arrow). However, few cells were seen per section due to the slow progression of rod death. (d-f) Progression of rod death determined by the reduction of the ONL as seen by HE staining. (g) End phase of rod death assessed by immunofluorescence with anti-rhodopsin. Although the ONL was reduced to one row of cells by PW35, no end point of rod death was determined. Rods continued to die slowly and even by PW70, many rods were still present (g). Interestingly, most of the rods at that age were confined to the ventral regions of the retina (see also FIG. 6). Age (in postnatal weeks (PW)) is indicated in the panels. Vertical bar in (al) indicates thickness of the ONL.

FIGS. 5a-5c depict summaries of the kinetics (a and b) and histological changes (c) that accompany rod and cone death across 4 mouse models. Red/green opsin protein levels were detectable mainly dorsally during cone degeneration (5c).

FIGS. 6a-6g depict dorsal cone death kinetics seen by the immunofluorescence with anti-red/green opsin as described in Example 1 as follows: (a-c) Loss of dorsal cones in the Rho-KO mutant over time as seen by the reduced expression of red-green opsin. (d, e) Loss of dorsal cones in the P23H mutant over time. (f, g) Higher magnification of a double staining with an antibody against red/green opsin (dark gray signal) and rhodopsin (light gray signal) showing that most rods that survived up to PW80 were in the ventral regions (g) of the retina whereas the red/green expressing cones were mostly dorsal (f).

FIGS. 7a-7c depict affymetrix microarray analysis as described in Example 1 as follows: (a) Equivalent time points in the 4 different mutants at which the microarray analysis was performed (R: approximately halfway through the major phase of rod death; C0: onset of cone death; C1 & C2 first and second time point during cone death respectively). Time is indicated in postnatal days (P) or postnatal weeks (PW). Cartoons depicting the progression of cone death are shown below the corresponding time points. (b) Distribution in percentage of the 195 genes that were annotated. (c) Distribution in percentage of the 68 genes (34.9%) that are part of metabolism in (b).

FIG. 8 depicts that red/green and blue opsin expression was not affected on the RNA level as described in Example 1 as follows: In situ hybridization for red/green opsin (first two rows) or blue opsin (third and fourth row) on retinal sections. RNA levels for red/green opsin and blue were comparable between ventral regions of mutant (first column), wild type animals treated with rapamycin (last column) or untreated wild type animals (second column).

FIGS. 9a-9m depict p*-mTOR in wild type and degenerating retinae as described in Example 1. All panels show immunofluorescence on retinal flat mounts (photoreceptor side up) with the exception of (b, c, g) which show retinal sections. Dark gray shows the nuclear DAPI stain. (a-c) p*-mTOR levels in wild type retinae. (a) Dorsal (up) enrichment of p*-mTOR. Higher magnification of dorsal and ventral region is shown to the right showing p*-mTOR in red and cone segments in light gray as detected by PNA. (b, c) Dorsal retinal sections stained for p*-mTOR (medium gray signal) and PNA (b) (green signal) or α-β-galactosidase (c) (light gray signal). The β-galactosidase is under the control of the human red/green opsin promoter and is expressed in all cones48 (see Material & Methods). The insets in (b, c) show higher magnification of the cone segments indicating that the p*-mTOR signal is located in the lower part of the outer segment (OS; IS: inner segment). (d-g) Rapamycin treatment of wild type mice leads to downregulation of red/green opsin ventrally (e) but not dorsally (d) (medium gray signal). Ventral blue opsin (f) (medium gray signal) remains unaffected, as does PNA (d-g) (light gray signal). Rapamycin treatment does also not affect mTOR phosphorylation in wild type (g) (dark gray signal). (h-m) Reduced levels of dorsal p*-mTOR during photoreceptor degeneration (medium graysignal). (h) Wild type control. (i, j) PDE-β mutant. The reduction starts during rod death at P15 (i) as the OSs (light gray signal: PNA) start to detach from the retinal pigmented epithelium. (i) By P30 only few cones (green signal: α-β-galactosidase) show high levels of p*-mTOR (red signal). (k-l) A similar reduction is seen in dorsal cones of the other three mutants (cones marked in light gray by PNA). (k) PDE-γ-KO P35. (l) Rho-KO PW20. (m) P23H PW70.

FIGS. 10a-10b depict the dependence of p*-mTOR levels on the presence of glucose as described in Example 1. Different media conditions were tested (a) during 4 hours of retinal explant culture. After culture, retinae were fixed and stained for p*-mTOR (light gray signal), PNA (medium gray signal) and DAPI (dark gray signal). Retinal flat mounts were imaged (b). Dorsal p*-mTOR was only detected when glucose was present in the media.

FIGS. 11a-11j depict the upregulation of Hif-1α and GLUT1 in cones as described in Example 1. All panels show immunofluorescent staining. Left column (a, d, g, h,) shows retinal flat mounts and right column (b, c, e, f, i, j) retinal sections. Dark gray shows nuclear DAPI staining and light gray shows cones marked with PNA. (a-f) Staining for HIF-1α (medium gray signal). (a) Wild type (PW10) (inset) showing higher magnification. (b, c) Cross sections in wild type (PW10). (c) DAPI overlap of (b). (d-f) During cone degeneration in PDE-β−/− (PW10) increased levels of HIF-1α are found in cones (d, inset). (e, f) Cross sections show that the increase of Hif-1α occurs mainly in cones (arrows point to cones that at this stage are located within the top layer of the inner nuclear layer). (f) DAPI overlap of (e). (g) GLUT1 expression in wild type (PW10) (medium gray signal). Most of the signal in between the cones reflects expression in rods. (h-j) Increased expression of GLUT1 in cones during degeneration seen in flat mounts (h) and sections (i-j). (i) Overlap of (j) with PNA.

FIGS. 12a-12h depict the upregulation of Hif-1α and GLUT1 in cones as described in Example 1. All panels show immunofluorescent signals within retinal sections. Dark gray shows nuclear DAPI staining and light gray shows cones marked with PNA. (a-d) Staining for HIF-1α (medium gray signal). (a) Wild-type at PW10 (see also FIG. 11a-c). (b) PDE-γ-KO at PW5. (c) Rho-KO at PW20. (d) P23H at PW70. (e-h) Staining for GLUT1 (red signal). (e) Wild-type at PW10. (f) PDE-γ-KO at PW5 with PNA overlap. (f′) Same image as (f) without PNA. (g) Rho-KO at PW20. (g′) Same image as (g) without PNA. (h) P23H at PW70. (h′) Same image as (h) without PNA.

White dotted line marks border between the ONL and INL.

FIGS. 13a-13d depict the increased levels of LAMP-2 at the lysosomal membrane as described in Example 1 as follows: (a-c) Immunofluorescence on retinal flat mounts where LAMP-2 is shown in light gray, red/green opsin in medium gray and dark gray signal shows nuclear DAPI stain. Insets in upper right corner (with box) show enlarged cells (arrow). (a) Wild type retinae at PW5 showing lysosome (small light gray dots) with normal LAMP-2 distribution. Weak red/green opsin signal is detected at the level of the PR nuclei since in wild type it is mainly found in the OSs. (b, c) PDE-β mutant at PW5. (b) Enlarged lysosomes (dots) due to accumulation of LAMP-2 at the lysosomal membrane are seen specifically in cones. (c) Confocal section of same field as in (b) taken at the level of the inner nuclear layer showing levels of LAMP-2 similar to those in wild type (a). (d) qRT-PCR for the 3 different LAMP-2 splice forms showing the relative concentration and the ratios between the PDE-β mutant and wild type.

FIGS. 14a-14m depict a retroviral vector, as described in Example 1, encoding a fusion protein between GFP and LC3 as used to infect the retinae of wild type (a-c) and PDE-β−/− (d-f) mice. Light gray signal shows expression of the fusion protein, medium gray signal shows red/green opsin expression, and dark gray signal shows nuclear DAPI staining. (a-f) Retinal flat mounts at PW10 showed uniform expression of the GFP fusion protein in cones without the formation of vesicular structures in wild type and mutant retinae. (a) DAPI overlap of (b). (c) 3D reconstruction of (b). Cone outer segments, as shown by red/green opsin signal (arrow), were attached to the cone inner segments (arrowhead), as shown by GFP signal. (d) DAPI overlap of (e). (f) Single confocal section showing cytoplasmic GFP and membrane bound red/green opsin (see also FIG. 3). FIG. 14 further depicts the levels of phosphorylated S6 (p*-S6) (medium gray signal, light gray signal marks cones with PNA, dark gray nuclear DAPI stain) in wild type (g, h) and mutant (i-m) cones. (g) Low levels of p*-S6 were seen in wild type cones but not in cone OSs. (h) DAPI overlap of (g). (i, j) Strong uniform expression in cones was seen the PDE-β mutant shortly after the end of the major rod death phase (PW3). Area in lower right corner shows a region where cones had started to die. (j) DAPI overlap of (i). (k-l) Higher magnification at PW5 showing same field at three different confocal depths. (k) Within the plane of the cone outer segments, high levels of p*-S6 were seen in cones when compared to segments of wild type cones. (l) Strong staining was also seen in the plane of the cone nuclei, indicating a uniform cytoplasmic distribution. (m) Within the plane of the INL, levels of p*-S6 were much lower than in cones.

FIGS. 15a-15h depict the effect of insulin levels on cone survival as set forth in Example 1 as follows: (a-c) Retinal flat mounts of PDE-β mutants at PW7 stained for lacZ (Wang, Y. et al. (1992) Neuron 9, 429-40; Punzo, C. & Cepko, C. (2007) Invest Ophthalmol Vis Sci 48, 849-57) to detect cones (see Material & Methods and FIG. 16). (a) Example of untreated control. (b) Example of mouse injected with streptozotocin. (c) Example of mouse injected daily with insulin. (d) Quantification of cone survival after 4 weeks of treatment. Data represents an average of at least 8 retinae and indicates on the y-axis percentage of cone surface area versus surface area of entire retina (see FIGS. 17 and 18). (e) Measurements of blood glucose levels and body weight (f) performed weekly over the time span of the experiment. (g, h) Immunofluorescent staining on retinal flat mounts for HIF-1α (medium gray signal) and PNA (light gray signal) in untreated control PDE-β^{-/-} (g) and PDE-β^{-/-} mice treated for 4 weeks with insulin (h). Dark gray shows nuclear DAPI.

FIGS. 16a-16f depict the cone-lacZ transgene in the PDE-β mutant at 7 weeks of age as described in Example 1 as follows: (a, b) Double labeling of cones with PNA (dark gray signal) and lacZ staining (light gray signal). More cones were labeled by lacZ than by PNA. Since PNA marks an extracellular matrix protein of the OS, once the OSs were reduced, PNA became a less reliable marker. (c, d) Double labeling of cones by α-red/green opsin (dark gray signal) and lacZ staining (X-gal; light gray signal) in the dorsal (c) and ventral (d) retina. Red/green opsin levels decreased ventrally during degeneration which made this marker not suitable for detection of cones across the retina. (e, f) Sections of retina stained for lacZ showing the signal in cones on top of the INL.

FIGS. 17a-17e depict a method to calculate cone survival as described in Example 1 as follows: (a-c) Show retinal flat mounts stained for lacZ (see FIG. 15). (a) Untreated control PDE-β−/− mouse at PW7. (b) PDE-β−/− mouse at PW7 treated with one injection of Streptozotocin at PW3. (c) PDE-β−/− mouse at PW7 treated for 4 weeks with daily injections of insulin starting at PW3. (a′-c′) Show inverted color images of corresponding panels (a-c). (a″-c″) Show only the green channels whereas (a′″-c′″) show only the red channels of the inverted color images (a′-c′). The red channel served as a proxy for the lacZ stain whereas the green channel served as a proxy for the retina. (d) Quantification of cone survival by calculating the surface area of red that co-localizes with green. Two different methods were employed, a fixed threshold and an adjusted threshold. The fixed threshold was determined by adjusting the lower intensity of the red channel in the image with the most intense lacZ staining (most intense red channel) to reflect the pattern of the lacZ staining. The same threshold for the red channel was then applied to all other images. As this method would under represent cone survival in mice that were not treated with insulin due to the less intense lacZ staining a second method was employed. For each image the lower intensity of the red channel was adjusted individually to match the blue pattern of the lacZ staining avoiding the problem of the difference in lacZ intensity. The increased intensity of lacZ in the insulin treated mice could be due to healthier cones that either have an increased transcription/translation or decreased protein degradation. (e) Shows the actual calculated values in percentage of cone survival for all retinae. Values are shown for the untreated mice, the Streptozotocin treated mice and the insulin treated mice. Values for both types of calculations are shown, for the fixed threshold and adjusted threshold.

FIGS. 18a-18c depict the assessment of cone survival after prolonged Insulin treatment as described in Example 1 as follows: (a) Composite of retinae after lacZ staining. First column shows untreated PDE-β^{-/-} mice at PW10. Second column shows retinae of PDE-β^{-/-} mice at PW10 that received a single injection of streptozotocin at PW3. Third column shows retinae of PDE-β^{-/-} mice at PW10 that received daily injections of insulin starting at PW3. (b) Shows quantification of cone survival by the two methods described in FIG. 17. There was no significant difference in cone survival between treated and untreated mice at PW10. (c) Shows comparison between the 4 and 7 weeks treatment.

FIG. 19 depicts an exemplary mTOR pathway.

FIG. 20 depicts a table of 230 genes that had statistically significant changes in all 4 mouse models and had fold changes >2 at the onset of cone death, when compared to the other three time points. The fold change is indicated as log₂. (AVG: Average of fold change from the 4 mutants; C0: Onset of cone death; R: peak of rod death; C1 & C2: first and second time point during cone death respectively, see also FIG. 7a).

### DETAILED DESCRIPTION OF THE INVENTION

The present invention is directed to the use of modulators of the mammalian target of rapamycine (mTOR) pathway for treating retinal disorders and, in particular, for prolonging the viability of a cone cell. The present invention is based, at least in part, on the discovery that a modulator of the mTOR pathway can be used to prolong the viability of a cone cell by decreasing and/or delaying cone cell death. Accordingly, the present invention provides methods for treating or preventing retinal disorders, e.g., retinitis pigmentosa, and for prolonging the viability of a cone cell, by contacting the cone cell with an mTOR modulator.

In addition, the present invention is directed to methods which involve increasing intracellular levels of glucose in a subject in order to treat retinal disorders, such as retinitis pigmentosa, in a subject and, further, to prolong the viability of a cone cell in a subject. In this regard, the present invention is based on the discovery that enhanced intracellular glucose levels can serve to prolong the viability of a cone cell by decreasing and/or delaying cone cell death. In various embodiments, a subject or isolated cell may be exposed to either glucose itself or glucose enhancers which serve to enhance the levels of intracellular glucose in the subject or the cell in order to achieve the desired therapeutic effect.

As used herein, the term “retinal disorders” refers generally to disorders of the retina. In one embodiment, the retinal disorder is associated with decreased viability, for example, death, of cone cells, and/ or rod cells. Moreover, in a particular embodiment, the retinal disorders of the present invention are not associated with blood vessel leakage and/or growth, for example, as is the case with diabetic retinopathy, but, instead are characterized primarily by reduced viability of cone cells and/ or rod cells. In certain embodiments, the retinal disorders are genetic disorders. In a particular embodiment, the retinal disorder is retinitis pigmentosa.

As used herein, the term “retinitis pigmentosa” is art known and encompasses a disparate group of genetic disorders of rods and cones. Retinal pigmentosa generally refers to retinal degeneration often characterized by the following manifestations: night blindness, progressive loss of peripheral vision, eventually leading to total blindness; ophthalmoscopic changes consist in dark mosaic-like retinal pigmentation, attenuation of the retinal vessels, waxy pallor of the optic disc, and in the advanced forms, macular degeneration. In some cases there can be a lack of pigmentation. Retinitis pigmentosa can be associated to degenerative opacity of the vitreous body, and cataract. Family history is prominent in retinitis pigmentosa; the pattern of inheritance may be autosomal recessive, autosomal dominant, or X-linked; the autosomal recessive form is the most common and can occur sporadically.

As used herein, the term “mTOR” or “mammalian target of rapamycine” refers to the art recognized serine/ threonine protein kinase involved in the regulation of cell growth, cell proliferation, cell motility, protein synthesis and transcription. mTOR further serves as a sensor of cellular nutrient status, energy status and redox status. As well known in the art, mTOR integrates input from multiple upstream pathways, including, but not limited to, insulin, growth factors (IGF-1 and IGF-2) and mitogens. Moreover, mTOR is known to operate as the catalytic subunit of two distinct molecular complexes in cells, i.e., mTOR Complex 1 (mTORC1) and mTOR Complex 2 (mTORC2).

Specifically, mTORC1 is composed of mTOR, regulatory associated protein of mTOR (Raptor), and mammalian LST8/G-protein β-subunit like protein (mLST8/GβL) and functions as a nutrient/energy/redox sensor and to control protein synthesis. The activity of this complex is stimulated by insulin, growth factors, serum, phosphatidic acid, amino acids (particularly leucine), and oxidative stress.

mTOR Complex 2 (mTORC2) is composed of mTOR, rapamycin-insensitive companion of mTOR (Rictor), GβL, and mammalian stress-activated protein kinase interacting protein 1 (mSIN1) and functions as an important regulator of the cytoskeleton through its stimulation of F-actin stress fibers, paxillin, RhoA, Rac1, Cdc42, and protein kinase C α (PKCα). In addition, mTORC2 phosphorylates the serine/threonine protein kinase Akt/PKB at a serine residue, thereby ultimately leading to full Akt activation.

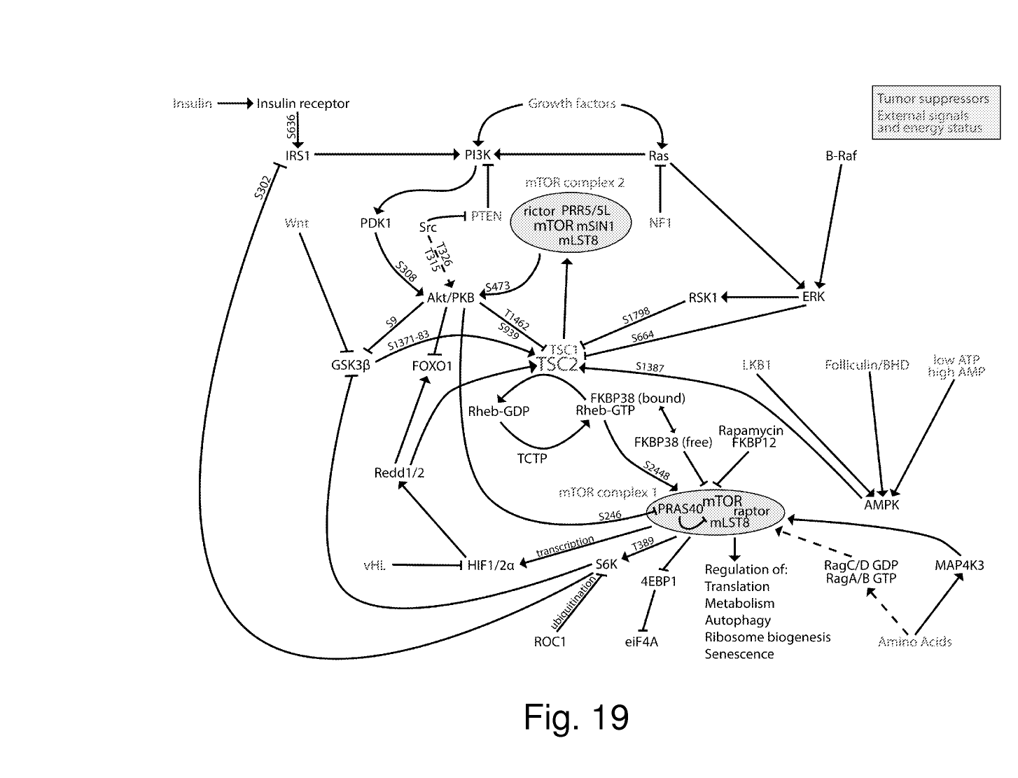

As used herein, the term “mTOR pathway” refers to biochemical pathways involving mTOR or mTOR complexes, for example, in the regulation of cell growth, cell proliferation, cell motility, protein synthesis and transcription and, further, the sensing of cellular nutrient status, energy status and redox status. In a particular embodiment, the mTOR pathway is as depicted in FIG. 19.

As used herein, the terms “mTOR modulator,” “modulator of mTOR,” “mTOR pathway modulator” or modulator of the mTOR pathway” refer to any moiety that modulates, for example, upregulates or downregulates, the activity, viability, presence, transcription, translation, and/or post-transcriptional or post-translational modification of mTOR and/or mTOR complex and/or that modify the activity and/or metabolic flux through the mTOR pathway and/or pathways upstream or downstream of the mTOR pathway. For example, such moieties include small molecules, proteins, amino acids, nucleic acid molecules, siRNA, aptamers, adnectins, antibodies or fragments thereof, growth factors, or hormones. Any of the art known mTOR modulators (e.g., one or more of the mTOR modulators described in PCT Publication No. WO 2008/027855, the contents of which are hereby incorporated herein by reference) may be used in the methods of the present invention.

In various embodiments, the mTOR modulator is selected from the group consisting of insulin, growth factors, IGF-1, IGF-2, mitogens, serum, phosphatidic acid, caffeic acid phenethyl ester (CAPE), amino acids, leucine, zinc and analogues or derivatives thereof. In a particular embodiment, the mTOR modulator is insulin. In certain embodiments, the mTOR modulator stimulates mTOR phosphorylation, e.g., Lysophosphatidic acid acyltransferase (LPAAT), e.g., LPAAT-theta. Alternatively or in addition, the mTOR modulator activates a receptor and/or a signal transduction cascade upstream of mTOR. In one embodiment of the invention, an mTOR modulator is a glucose enhancer. In one embodiment, an mTOR modulator acts through the insulin receptor.

As used herein, the term “glucose enhancer” refers to any moiety that increases the level of intracellular glucose. For example, such moieties include small molecules, proteins, amino acids, nucleic acid molecules, siRNA, aptamers, adnectins, antibodies or fragments thereof, growth factors, or hormones. In various embodiments, the glucose enhancers may serve to modulate biochemical pathways leading to enhanced intracellular glucose levels. In one embodiment, the glucose enhancers may serve to increase glucose uptake into cells by, for example, enhancing the activity or levels of glucose transporters such as GLUT-1, GLUT-2, GLUT-3, GLUT-4 and/or GLUT-5 glucose transporters. In one embodiment, a glucose enhancer is a nucleic acid molecule encoding a glucose transporter protein cloned into a recombinant expression vector, e.g., a viral vector, for use in gene therapy.

As used herein, the term “subject” includes warm-blooded animals, preferably mammals, including humans. In a preferred embodiment, the subject is a primate. In an even more preferred embodiment, the primate is a human.

As used herein, the various forms of the term “modulate” are intended to include stimulation (e.g., increasing or upregulating a particular response or activity) and inhibition (e.g., decreasing or downregulating a particular response or activity).

As used herein, the term “contacting” (e.g., contacting a cell with an agent, such as an mTOR modulator, glucose, and/or glucose enhancer) is intended to include incubating the agent and the cell together in vitro (e.g., adding the agent to cells in culture) or administering the agent to a subject such that the agent and cells of the subject are contacted in vivo. The term “contacting” is not intended to include exposure of cells to an agent that may occur naturally in a subject (i.e., exposure that may occur as a result of a natural physiological process).

As used herein, the term “administering” to a subject includes dispensing, delivering or applying an mTOR modulator, glucose, and/or a glucose enhancer to a subject by any suitable route for delivery of the mTOR modulator, glucose, and/or a glucose enhancer to the desired location in the subject, including delivery by intraocular administration or intravenous administration. Alternatively or in combination, delivery is by the topical, parenteral or oral route, intracerebral injection, intramuscular injection, subcutaneous/intradermal injection, buccal administration, transdermal delivery and administration by the rectal, colonic, vaginal, intranasal or respiratory tract route.

As used herein, the term “effective amount” includes an amount effective, at dosages and for periods of time necessary, to achieve the desired result, e.g., sufficient to treat a subject suffering from a retinal disorder, for example, retinitis pigmentosa; sufficient to prevent a retinal disorder, for example, in a subject likely to develop the retinal disorder; or sufficient to prolong the viability of a cone cell. An effective amount of an mTOR modulator, glucose, and/or glucose enhancer, as defined herein may vary according to factors such as the state, severity and extent of the condition, e.g., abnormal cone cell death or a retinal disorder such as retinitis pigmentosa, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the mTOR modulator, glucose, and/or glucose enhancer are outweighed by the therapeutically beneficial effects.

Various additional aspects of the methods of the invention are described in further detail in the following subsections.

### I. Methods of the Invention

The present invention provides methods for treating or preventing a retinal disorder in a subject. The methods include administering to the subject an mTOR modulator in an amount effective for modulating mTOR activity in the subject, thereby treating or preventing a retinal disorder in the subject.

The present invention also provides methods for treating or preventing retinitis pigmentosa in a subject. The methods generally comprise administering to the subject an mTOR modulator in an amount effective for modulating mTOR activity in the subject, thereby treating or preventing retinitis pigmentosa in the subject.

The present invention further provides methods for prolonging the viability of a cone cell. The methods generally comprise contacting the cell with an mTOR modulator in an amount effective for modulating mTOR activity in the cone cell, thereby prolonging the viability of the cone cell.

In one embodiment, the viability or survival of a cone cell is prolonged for e.g., about 1 week, about 2 weeks, about 3 weeks, about 4 weeks, about 5 weeks, about 6 weeks, about 7 weeks, about 8 weeks, about 3 months, about 6 months, about 1 year, about 2 years, about 3 years, about 4 years, about 5 years, about 10 years, about 15, years, about 20 years, about 25 years, about 30 years, about 40 years, about 50 years, about 60 years, about 70 years, or about 80 years. Times intermediate to the above-recited times are also contemplated by the invention.

In another aspect, the present invention provides methods for treating or preventing a retinal disorder in a subject. Such methods generally comprise administering to the subject an agent which enhances the intracellular levels of glucose, thereby treating or preventing a retinal disorder in a subject.

The present invention also provides methods for treating or preventing retinitis pigmentosa in a subject by administering to the subject an agent which enhances the intracellular levels of glucose, thereby treating or preventing retinitis pigmentosa in the subject.

The present invention further provides methods for prolonging the viability of a cone cell by contacting the cell with an agent which enhances the intracellular levels of glucose in the cone cell, thereby prolonging the viability of the cone cell.

The viability or survival of a cone cell may be prolonged for e.g., about 1 week, about 2 weeks, about 3 weeks, about 4 weeks, about 5 weeks, about 6 weeks, about 7 weeks, about 8 weeks, about 3 months, about 6 months, about 1 year, about 2 years, about 3 years, about 4 years, about 5 years, about 10 years, about 15, years, about 20 years, about 25 years, about 30 years, about 40 years, about 50 years, about 60 years, about 70 years, or about 80 years. Times intermediate to the above-recited times are also contemplated by the invention.

Without intending to be bound by theory, it is believed that the mTOR modulators, glucose and/or glucose enhancers serve to increase nutrient levels, for example, intracellular glucose, to increase glucose uptake by cells, to increase membrane synthesis or the rate thereof, to increase the synthesis of phospholipids, to increase metabolic flux through the pentose phosphate cycle and/ or to increase intracellular generation of NADPH. By doing so, the methods of the present invention serve to increase the viability of cone cells and to treat and/or prevent retinal disorders such as retinitis pigmentosa.

mTOR modulators suitable for use in the methods of the invention include, for example, insulin, growth factors, IGF-1, IGF-2, mitogens, serum, phosphatidic acid, caffeic acid phenethyl ester (CAPE), amino acids, leucine, zinc and analogues or derivatives thereof. In a particular embodiment, the mTOR modulator is insulin. In certain embodiments, the mTOR modulator stimulates mTOR phosphorylation, e.g., Lysophosphatidic acid acyltransferase (LPAAT), e.g., LPAAT-theta. Alternatively or in addition, the mTOR modulator activates a receptor and/or a signal transduction cascade upstream of mTOR. In one embodiment, the mTOR modulator is a glucose enhancer.

In various embodiments, an agent that enhances glucose levels may serve to modulate biochemical pathways leading to enhanced intracellular glucose levels. In one embodiment, the agent that enhances the activity or level of intracellular glucose is a glucose transporter. In one embodiment, the agent is a nucleic acid molecule. For example, glucose transporters for use in the present invention may belong to the GLUT family of transporters (including at least one of GLUT1, GLUT2, GLUT3, GLUT4, GLUT5, GLUT6, GLUT7, GLUT8, GLUT9, GLUT10, GLUT11, GLUT12 and GLUT13), encoded by the SLC2 family of genes (including at least one of SLC2A1, SLC2A2, SLC2A3, SLC2A4, SLC2A5, SLC2A6, SLC2A7, SLC2A8, SLC2A9, SLC2A10, SLC2A11, SLC2A12 and SLC2A13).

In one embodiment of the invention, a glucose enhancer for use in the methods of the invention is a nucleic acid molecule encoding a glucose transporter. For example, a cDNA (full length or partial cDNA sequence) may be cloned into a recombinant expression vector used as a gene therapy vector, and the vector may be transfected into cells using standard molecular biology techniques. The cDNA can be obtained, for example, by amplification using the polymerase chain reaction (PCR) or by screening an appropriate cDNA library.

The nucleic acids for use in the methods of the invention can also be prepared, e.g., by standard recombinant DNA techniques. A nucleic acid of the invention can also be chemically synthesized using standard techniques. Various methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

In one embodiment, a nucleic acid molecule encoding an mTOR modulator or a glucose enhancer, e.g., a glucose transporter, may be present in an inducible construct. In another embodiment, a nucleic acid molecule encoding an mTOR modulator or a glucose enhancer may be present in a construct which leads to constitutive expression. In one embodiment, a nucleic acid molecule encoding an mTOR modulator or a glucose enhancer may be delivered to cells, or to subjects, in the absence of a vector.

A nucleic acid molecule encoding an mTOR modulator or a glucose enhancer may be delivered to cells or to subjects using a viral vector, preferably one whose use for gene therapy is well known in the art. Techniques for the formation of vectors or virions are generally described in “Working Toward Human Gene Therapy,” Chapter 28 in Recombinant DNA, 2nd Ed., Watson, J. D. et al., eds., New York: Scientific American Books, pp. 567-581 (1992). An overview of suitable viral vectors or virions is provided in Wilson, J. M., Clin. Exp. Immunol. 107(Suppl. 1):31-32 (1997), as well as Nakanishi, M., Crit. Rev. Therapeu. Drug Carrier Systems 12:263-310 (1995); Robbins, P. D., et al., Trends Biotechnol. 16:35-40 (1998); Zhang, J., et al., Cancer Metastasis Rev. 15:385-401(1996); and Kramm, C. M., et al., Brain Pathology 5:345-381 (1995). Such vectors may be derived from viruses that contain RNA (Vile, R. G., et al., Br. Med Bull. 51:12-30 (1995)) or DNA (Ali M., et al., Gene Ther. 1:367-384 (1994)).

Examples of viral vector systems utilized in the gene therapy art and, thus, suitable for use in the present invention, include the following: retroviruses (Vile, R. G., supra; U.S. Pat. Nos. 5,741,486 and 5,763,242); adenoviruses (Brody, S. L., et al., Ann. N.Y. Acad. Sci. 716: 90-101 (1994); Heise, C. et al., Nat. Med. 3:639-645 (1997)); adenoviral/retroviral chimeras (Bilbao, G., et al., FASEB J. 11:624-634 (1997); Feng, M., et al., Nat. Biotechnol. 15:866-870 (1997)); adeno-associated viruses (Flotte, T. R. and Carter, B. J., Gene Ther. 2:357-362 (1995); U.S. Pat. No. 5,756,283); herpes simplex virus I or II (Latchman, D. S., Mol. Biotechnol. 2:179-195 (1994); U.S. Pat. No. 5,763,217; Chase, M., et al., Nature Biotechnol. 16:444-448 (1998)); parvovirus (Shaughnessy, E., et al., Semin Oncol. 23:159-171 (1996)); reticuloendotheliosis virus (Donburg, R., Gene Therap. 2:301-310 (1995)). Extrachromosomal replicating vectors may also be used in the gene therapy methods of the present invention. Such vectors are described in, for example, Calos, M. P. (1996) Trends Genet. 12:463-466, the entire contents of which are incorporated herein by reference. Other viruses that can be used as vectors for gene delivery include poliovirus, papillomavirus, vaccinia virus, lentivirus, as well as hybrid or chimeric vectors incorporating favorable aspects of two or more viruses (Nakanishi, M. (1995) Crit. Rev. Therapeu. Drug Carrier Systems 12:263-310; Zhang, J., et al. (1996) Cancer Metastasis Rev. 15:385-401; Jacoby, D. R., et al. (1997) Gene Therapy 4:1281-1283).

The term “AAV vector” refers to a vector derived from an adeno-associated virus serotype, including without limitation, AAV-1, AAV-2, AAV-3, AAV-4, AAV-5, or AAVX7. “rAAV vector” refers to a vector that includes AAV nucleotide sequences as well as heterologous nucleotide sequences. rAAV vectors require only the 145 base terminal repeats in cis to generate virus. All other viral sequences are dispensable and may be supplied in trans (Muzyczka (1992) Curr. Topics Microbiol. Immunol. 158:97). Typically, the rAAV vector genome will only retain the inverted terminal repeat (ITR) sequences so as to maximize the size of the transgene that can be efficiently packaged by the vector. The ITRs need not be the wild-type nucleotide sequences, and may be altered, e.g., by the insertion, deletion or substitution of nucleotides, as long as the sequences provide for functional rescue, replication and packaging. In particular embodiments, the AAV vector is an AAV2/5 or AAV2/8 vector. Suitable AAV vectors are described in, for example, U.S. Pat. No. 7,056,502 and Yan et al. (2002) J. Virology 76(5):2043-2053, the entire contents of which are incorporated herein by reference.

As used herein, the term “lentivirus” refers to a group (or genus) of retroviruses that give rise to slowly developing disease. Viruses included within this group include HW (human immunodeficiency virus; including but not limited to HW type 1 and HW type 2), the etiologic agent of the human acquired immunodeficiency syndrome (AIDS); visna-maedi, which causes encephalitis (visna) or pneumonia (maedi) in sheep; the caprine arthritis-encephalitis virus, which causes immune deficiency, arthritis, and encephalopathy in goats; equine infectious anemia virus (EIAV), which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SW), which cause immune deficiency and encephalopathy in sub-human primates. Diseases caused by these viruses are characterized by a long incubation period and protracted course. Usually, the viruses latently infect monocytes and macrophages, from which they spread to other cells. HW, FW, and SW also readily infect T lymphocytes (i.e., T-cells). In one embodiment of the invention, the lentivirus is not HIV.

As used herein, the term “adenovirus” (“Ad”) refers to a group of double-stranded DNA viruses with a linear genome of about 36 kb. See, e.g., Berkner et al., Curr. Top. Microbiol. Immunol., 158: 39-61 (1992). In some embodiments, the adenovirus-based vector is an Ad-2 or Ad-5 based vector. See, e.g., Muzyczka, Curr. Top. Microbiol. Immunol., 158: 97-123, 1992; Ali et al., 1994 Gene Therapy 1: 367-384; U.S. Pat. Nos. 4,797,368, and 5,399,346. Suitable adenovirus vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types. Additionally, introduced adenovirus DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenovirus genome for foreign DNA is large (up to 8 kilobases) relative to other gene delivery vectors (Haj-Ahmand et al. J. Virol. 57, 267-273 [1986]).

In one embodiment, an adenovirus is a replication defective adenovirus. Most replication-defective adenoviral vectors currently in use have all or parts of the viral E1 and E3 genes deleted but retain as much as 80% of the adenovirus genetic material. Adenovirus vectors deleted for all viral coding regions are also described by Kochanek et al. and Chamberlain et al. (U.S. Pat. No. 5,985,846 and U.S. Pat. No. 6,083,750). Such viruses are unable to replicate as viruses in the absence of viral products provided by a second virus, referred to as a “helper” virus.

In one embodiment, an adenoviral vector is a “gutless” vector. Such vectors contain a minimal amount of adenovirus DNA and are incapable of expressing any adenovirus antigens (hence the term “gutless”). The gutless replication defective Ad vectors provide the significant advantage of accommodating large inserts of foreign DNA while completely eliminating the problem of expressing adenoviral genes that result in an immunological response to viral proteins when a gutless replication defective Ad vector is used in gene therapy. Methods for producing gutless replication defective Ad vectors have been described, for example, in U.S. Pat. No. 5,981,225 to Kochanek et al., and U.S. Pat. Nos. 6,063,622 and 6,451,596 to Chamberlain et al; Parks et al., PNAS 93:13565 (1996) and Lieber et al., J. Virol. 70:8944-8960 (1996).

In another embodiment, an adenoviral vector is a “conditionally replicative adenovirus” (“CRAds”). CRAds are genetically modified to preferentially replicate in specific cells by either (i) replacing viral promoters with tissue specific promoters or (ii) deletion of viral genes important for replication that are compensated for by the target cells only. The skilled artisan would be able to identify epithelial cell specific promoters.

Other art known adenoviral vectors may be used in the methods of the invention. Examples include Ad vectors with recombinant fiber proteins for modified tropism (as described in, e.g., van Beusechem et al., 2000 Gene Ther. 7: 1940-1946), protease pre-treated viral vectors (as described in, e.g., Kuriyama et al., 2000 Hum. Gene Ther. 11: 2219-2230), E2a temperature sensitive mutant Ad vectors (as described in, e.g., Engelhardt et al., 1994 Hum. Gene Ther. 5: 1217-1229), and “gutless” Ad vectors (as described in, e.g., Armentano et al., 1997 J. Virol. 71: 2408-2416; Chen et al., 1997 Proc. Nat. Acad. Sci. USA 94: 1645-1650; Schieder et al., 1998 Nature Genetics 18: 180-183).

In a particular embodiment, the viral vector for use in the methods of the present invention is an AAV vector. In particular embodiments, the viral vector is an AAV2/5 or AAV2/8 vector. Such vectors are described in, for example, U.S. Pat. No. 7,056,502, the entire contents of which are incorporated herein by reference.

In one embodiment, an LIA retrovirus may be used to deliver nucleic acids encoding an mTOR modulator or a glucose enhancer (Cepko et al. (1998) Curr. Top. Dev. Biol. 36:51; Dyer and Cepko (2001) J. Neurosci. 21:4259). The viral titer may be varied to alter the expression levels. The viral titer may be in any suitable range. For example, the viral titer may range from about 10⁶ cfu/ml to 10⁸ cfu/ml. The amount of virus to be added may also be varied. The volume of virus, or other nucleic acid and reagent, added can be in any suitable range.

The vector will include one or more promoters or enhancers, the selection of which will be known to those skilled in the art. Suitable promoters include, but are not limited to, the retroviral long terminal repeat (LTR), the SV40 promoter, the human cytomegalovirus (CMV) promoter, and other viral and eukaryotic cellular promoters known to the skilled artisan.

Guidance in the construction of gene therapy vectors and the introduction thereof into affected subjects for therapeutic purposes may be obtained in the above-referenced publications, as well as in U.S. Pat. Nos. 5,631,236, 5,688,773, 5,691,177, 5,670,488, 5,529,774, 5,601,818, and PCT Publication No. WO 95/06486, the entire contents of which are incorporated herein by reference.

Generally, methods are known in the art for viral infection of the cells of interest. The virus can be placed in contact with the cell of interest or alternatively, can be injected into a subject suffering from a retinal disorder, for example, as described in United States Provisional Patent Application No. 61/169,835 and PCT Application No. PCT/US09/053730, the contents of each of which are incorporated by reference.

Gene therapy vectors comprising, an mTOR modulator or a glucose enhancer, e.g., a glucose transporter, can be delivered to a subject or a cell by any suitable method in the art, for example, intravenous injection (e.g., intravitreal or subretinal injection), local administration, e.g., pplication of the nucleic acid in a gel, oil, or cream, (see, e.g., U.S. Pat. No. 5,328,470), stereotactic injection (see, e.g., Chen et al. (1994) Proc. Natl. Acad. Sci. U.S.A. 91:3054), gene gun, or by electroporation (see, e.g., Matsuda and Cepko (2007) Proc. Natl. Acad. Sci. U.S.A. 104:1027), using lipid-based transfection reagents, or by any other suitable transfection method.

As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, Calif.), LIPOFECTAMINE® (Invitrogen), FUGENE® (Roche Applied Science, Basel, Switzerland), JETPEI™ (Polyplus-transfection Inc., New York, N.Y.), EFFECTENE® (Qiagen, Valencia, Calif.), DREAMFECT™ (OZ Biosciences, France) and the like), or electroporation (e.g., in vivo electroporation). Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals.

In one embodiment, an mTOR modulator or glucose enhancer is delivered to a subject or cells in the form of a peptide or protein. In order to produce such peptides or proteins, recombinant expression vectors of the invention can be designed for expression of one or more mTOR modulator proteins and/or glucose enhancer proteins, e.g., glucose transporter proteins, and/or portion(s) thereof in prokaryotic or eukaryotic cells. For example, one or more glucose transporter proteins and/or portion(s) thereof can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

In one embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include retinal cell-type-specific promoters (e.g., rhodopsin regulatory sequences, Cabp5, Cralbp, Nr1, Crx, Ndrg4, clusterin, Rax, Hes1 and the like (Matsuda and Cepko, supra)), the albumin promoter (liver-specific, Pinkert et al. (1987) Genes Dev. 1:268), neuron-specific promoters (e.g., the neurofilament promoter; Byrne and Ruddle (1989) Proc. Natl. Acad. Sci. U.S.A. 86:5473). Developmentally-regulated promoters are also encompassed, for example the a-fetoprotein promoter (Campes and Tilghman (1989) Genes Dev. 3:537).

In certain embodiments, the methods of the present invention involve co-administration of multiple mTOR modulators or a pharmaceutically acceptable salt thereof, for example, at least two of mTOR modulators selected from the group consisting of insulin, growth factors, IGF-1, IGF-2, mitogens, serum, phosphatidic acid, amino acids, leucine and analogues or derivatives thereof. Alternatively, mTOR modulators may be administered in combination with glucose and/or glucose enhancers.

The methods described herein can be performed in vitro. For example, mTOR activity and/or intracellular glucose levels can be modulated in a cell in vitro and then the treated cells can be administered or re-administered to a subject. For practicing the methods in vitro, cells (e.g., mammalian cells, such as human cells) can be obtained from a subject by standard methods and incubated (e.g., cultured) in vitro with an agent which modulates mTOR and/or enhances intracellular glucose levels. Methods for isolating cells are well known in the art. The cells can be re-administered to the same subject, or another subject which is compatible with the donor of the cells.

For administration of cells to a subject, it may be preferable to first remove residual agents in the culture from the cells before administering them to the subject. This can be done, for example, by gradient centrifugation of the cells or by washing of the tissue. Methods for the ex vivo genetic modification of cells followed by re-administration to a subject are well known in the art and described in, for example, U.S. Pat. No. 5,399,346 the entire contents of which are incorporated herein by reference.

The claimed methods of modulation are not meant to include naturally occurring events. For example, the term “agent” or “modulator” is not meant to embrace endogenous mediators produced by the cells of a subject.

Application of the methods of the invention for the treatment and/or prevention of a retinal disorder can result in curing the disorder, decreasing at least one symptom associated with the disorder, either in the long term or short term or simply a transient beneficial effect to the subject. Accordingly, as used herein, the terms “treat,” “treatment” and “treating” include the application or administration of agents, as described herein, to a subject who is suffering from a retinal disorder, or who is susceptible to such conditions with the purpose of curing, healing, alleviating, relieving, altering, remedying, ameliorating, improving or affecting such conditions or at least one symptom of such conditions. As used herein, the condition is also “treated” if recurrence of the condition is reduced, slowed, delayed or prevented.

Subjects suitable for treatment using the regimens of the present invention should have or are susceptible to developing a retinal disorder. For example, subjects may be genetically predisposed to development of a retinal disorder. Alternatively, abnormal progression of the following factors including, but not limited to visual acuity, the rate of death of cone and/or rod cells, night vision, peripheral vision, attenuation of the retinal vessels, and other ophthalmoscopic factors associated with retinal disorders such as retinitis pigmentosa may indicate the existence of or a predisposition to a retinal disorder. Other art recognized symptoms or risk factors may be monitored using methods well known in the art.

In one embodiment of the invention, the retinal disorder is retinitis pigmentosa. In another embodiment, the retinal disorder is associated with decreased viability of cone cells. In yet another embodiment, the retinal disorder is associated with decreased viability of rod cells. In one embodiment, the retinal disorder is not diabetic retinopathy. In another embodiment, the retinal disorder is not associated with blood vessel leakage and/or blood vessel growth.

The mTOR modulators, glucose, and/or glucose enhancers, as described herein, may be administered as necessary to achieve the desired effect and depend on a variety of factors including, but not limited to, the severity of the condition, age and history of the subject and the nature of the composition, for example, the identity of the genes or the affected biochemical pathway. In various embodiments, the mTOR modulators, glucose, and/or glucose enhancers may be administered at least two, three, four, five or six times a day. Additionally, the therapeutic or preventative regimens may cover a period of at least about 2, 3, 4, 5, 6, 7, 8, 9, 10, 11, 12, 13, 14, 15, 16, 17, 18, 19, 20, 21, 22, 23, 24 weeks, 3 months, 6 months, 1 year, 2 years, 3 years, 4 years, 5 years, 10 years, 15 years, 20 years, 30 years, 40 years, 50 years, 60 years, 70 years, or 80 years. Times intermediate to the above-recited times are also contemplated by the invention.

The ability of an agent to modulate the activity of mTOR and/or intracellular levels of glucose can be determined as described herein, e.g., by determining the ability of the agent to modulate: cell viability (e.g., modulation of apoptosis), cleavage of LaminA or Caspase 3; expression of Opnlsw, Opnlmw, LAMP-2A, LAMP-2B, or LAMP-2C; protein production of LAMP-2A, LAMP-2B, LAMP-2C, HIF1-α, or GLUT1; phosphorylation of mTOR, S6K1, AMPK, PTEN, or Akt; phospholipid production; production of reactive oxygen species; and/or the expression and protein synthesis of photoreceptor specific opsins. The assays described in the Examples section below may also be used to determine whether an agent modulates the activity of mTOR and/or intracellular levels of glucose.

In various embodiments, the methods of the present invention further comprise monitoring the effectiveness of treatment. For example, visual acuity, the rate of death of cone and/ or rod cells, night vision, peripheral vision, attenuation of the retinal vessels, and other ophthalmoscopic changes associated with retinal disorders such as retinitis pigmentosa may be monitored to assess the effectiveness of treatment. The assays described in the Examples section below may also be used to monitor the effectiveness of treatment.

### II. Pharmaceutical Compositions for Use in the Methods of the Invention

The mTOR modulators, glucose and/or glucose enhancers used in the methods of the present invention may be incorporated into pharmaceutical compositions suitable for administration to a subject, which may, for example, allow for sustained delivery of the active agent for a period of at least several weeks to a month or more. Preferably, the mTOR modulator, glucose and/or glucose enhancer is the only active ingredient(s) formulated into the pharmaceutical composition, although in certain embodiments the mTOR modulator, glucose and/or glucose enhancer may be combined with one or more other active ingredients including, for example, modulators of pathways upstream or downstream of the mTOR pathway. In addition, at least two of the mTOR modulator, glucose and/or glucose enhancer may be present in the composition. Other pharmaceutically active compounds that may be used can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference.

In one embodiment, the pharmaceutical composition comprises an mTOR modulator, glucose and/or glucose enhancer and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. In one embodiment, the carrier is suitable for intraocular, parenteral, intravenous, intraperitoneal, topical, or intramuscular administration. In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

The pharmaceutical compositions of the present invention may be administered in the form of injectable compositions which can be prepared either as liquid solutions or suspensions. The pharmaceutical compositions may also be emulsified. Suitable excipients for use in such compositions are, for example, water, saline, dextrose, glycerol, or ethanol, and combinations thereof. In addition, if desired, the pharmaceutical compositions may contain minor amounts of auxiliary substances such as wetting or emulsifying agents, pH-buffering agents, adjuvants or immunopotentiators.

In a particular embodiment, the mTOR modulator, glucose and/or glucose enhancer is incorporated in a composition suitable for intraocular administration. For example, the compositions may be designed for intravitreal, subconjuctival, sub-tenon, periocular, retrobulbar, suprachoroidal, and/or intrascleral administration, for example, by injection, to effectively treat the retinal disorder. Additionally, a sutured or refillable dome can be placed over the administration site to prevent or to reduce “wash out”, leaching and/or diffusion of the active agent in a non-preferred direction.

Relatively high viscosity compositions, as described herein, may be used to provide effective, and preferably substantially long-lasting delivery of an mTOR modulator, glucose and/or glucose enhancer, for example, by injection to the posterior segment of the eye. A viscosity inducing agent can serve to maintain the mTOR modulator, glucose and/or glucose enhancer in a desirable suspension form, thereby preventing deposition of the composition and the mTOR modulator in the bottom surface of the eye. Such compositions can be prepared as described in U.S. Patent No. 5,292,724, the contents of which are hereby incorporated herein by reference.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, monostearate salts and gelatin. Moreover, the compounds of the invention can be administered in a time release formulation, for example in a composition which includes a slow release polymer. The mTOR modulator, glucose and/or glucose enhancer compositions can be prepared with carriers that will protect the active agent against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the mTOR modulator, glucose and/or glucose enhancer in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The pharmaceutical compositions of the invention can be formulated with one or more additional compounds that enhance the solubility of the mTOR modulator, glucose and/or glucose enhancer. Preferred compounds to be added to formulations to enhance the solubility of the mTOR modulator, glucose and/or glucose enhancer are cyclodextrin derivatives, preferably hydroxypropyl-γ-cyclodextrin. For example, inclusion in the formulation of hydroxypropyl-γ-cyclodextrin at a concentration 50-200 mM may increase the aqueous solubility of the active agent.

Another formulation for the mTOR modulator, glucose and/or glucose enhancer comprises the detergent Tween-80, polyethylene glycol (PEG) and ethanol in a saline solution. A non-limiting example of such a preferred formulation is 0.16% Tween-80, 1.3% PEG-3000 and 2% ethanol in saline.

In one embodiment, the mTOR modulator, glucose and/or glucose enhancer composition is administered to the subject as a sustained-release formulation using a pharmaceutical composition comprising a solid ionic complex of mTOR modulator, glucose and/or glucose enhancer and a carrier macromolecule, wherein the carrier and the active agent used to form the complex are combined at a weight ratio of carrier: active agent of, for example, 0.5:1 to 0.1:1. In other embodiments, the carrier and active agent used to form the complex are combined at a weight ratio of carrier: active agent of 0.8:1, 0.7:1, 0.6:1, 0.5:1, 0.4:1, 0.3:1, 0.25:1, 0.2:1, 0.15:1, or 0.1:1. Ranges intermediate to the above recited values, e.g., 0.8:1 to 0.4:1, 0.6:1 to 0.2:1, or 0.5:1 to 0.1:1 are also intended to be part of this invention. For example, ranges of values using a combination of any of the above recited values as upper and/or lower limits are intended to be included.

In another embodiment, the mTOR modulator, glucose and/or glucose enhancer is administered to the subject using a pharmaceutical composition comprising a solid ionic complex of the mTOR modulator, glucose and/or glucose enhancer and a carrier macromolecule, wherein the mTOR modulator, glucose and/or glucose enhancer content of the complex is at least 0.05% by weight, preferably at least 0.05%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95% or 1.00% w/w. Ranges intermediate to the above recited amounts, e.g., about 0.08% to about 0.73% w/w, are also intended to be part of this invention. For example, ranges of values using a combination of any of the above recited values as upper and/or lower limits are intended to be included.

As used herein, the term “sustained delivery” or “sustained release” is intended to refer to continual delivery of mTOR modulator, glucose and/or glucose enhancer in vivo over a period of time following administration, preferably at least several days, a week or several weeks and up to a month or more. In a preferred embodiment, a formulation of the invention achieves sustained delivery for at least about 7, 14, 21 or 28 days, at which point the sustained release formulation can be re-administered to achieve sustained delivery for another 28 day period (which re-administration can be repeated every 7, 14, 21 or 28 days to achieve sustained delivery for several months to years). Sustained delivery of the mTOR modulator, glucose and/or glucose enhancer can be demonstrated by, for example, the continued therapeutic effect of the active agent over time. Alternatively, sustained delivery of the mTOR modulator, glucose and/or glucose enhancer may be demonstrated by detecting the presence of the active agent in vivo over time.

In another embodiment, the mTOR modulator, glucose and/or glucose enhancer is incorporated into a composition suitable for oral administration. Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: A binder such as microcrystalline cellulose, gum tragacanth or gelatin; an excipient such as starch or lactose, a disintegrating agent such as alginic, acid, Primogel, or corn starch; a lubricant such as magnesium stearate or Sterotes; a glidant: such as colloidal silicon dioxide; a sweetening agent such as sucrose or saccharin; or a flavoring agent such as peppermint, methyl salicylate, or orange flavoring.

In one embodiment, mTOR modulators, glucose and/or glucose enhancers described herein are prepared with carriers that will protect the compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811.

Systemic administration of an mTOR modulator, glucose, and/or glucose enhancer may also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical formulation contains an effective amount of the mTOR modulator, glucose and/or glucose enhancer. An effective amount of an mTOR modulator, glucose and/or glucose enhancer, as defined herein may vary according to factors such as the state, severity and extent of the condition, e.g., abnormal cone cell death or a retinal disorder such as retinitis pigmentosa, age, and weight of the subject, and the ability of the compound to elicit a desired response in the subject. Dosage regimens may be adjusted to provide the optimum therapeutic response. An effective amount is also one in which any toxic or detrimental effects of the mTOR modulator, glucose and/or glucose enhancer are outweighed by the therapeutically or prophylactically beneficial effects.

This invention is further illustrated by the following examples which should not be construed as limiting. The contents of all references, patents and published patent applications cited throughout this application, as well as the Figures and Sequence Listing, are hereby incorporated by reference.

### EXAMPLES

### Materials and Methods

Animals: Wild type (wt) mice (C57B1/6N) and PDE-β−/− mice (normally referred as rdl or FVB/N) were purchased from Taconic Farms. The PDE-β−/− mice have a mutation in the β-subunit of cGMP phosphodiesterase (Bowes, C. et al. (1990) Nature 347, 677-80) (PDE). The PDE-γ knock-out (PDE-γ-KO) lacks the y-subunit of PDE and was provided by Steve Tsang (Tsang, S. H. et al. (1996) Science 272, 1026-9) (UCLA). The rhodopsin knock-out (Rho-KO) lacks the rod-specific opsin gene and was provided by Janis Lem (Tsang, S. H. et al. (1996) Science 272, 1026-9; Lem, J. et al. (1999) Proc Natl Acad Sci USA 96, 736-41) (Tufts Medical School). The P23H mouse has a proline-23 to histidine mutation in the rhodopsin gene and was provided by Muna Naash (Naash, M. I., et al. (19903) Proc Natl Acad Sci USA 90, 5499-503) (University of Oklahoma). As this mouse carries a transgene the strain was always crossed back to C57B1/6N to ensure that none of the progeny would carry two alleles of the transgene. The transgene is specifically expressed in rods (Gouras, P., et al. (1994) Vis Neurosci 11, 1227-31; Woodford, B. J., et al. (1994) Exp Eye Res 58, 631-5; al-Ubaidi, M. R. et al. (1990) J Biol Chem 265, 20563-9) and carries 3 mutations in the rhodopsin gene (Val-20 to Gly, Pro-23 to His, Pro-27 to Leu). In this study it is referred as the P23H mutant. The cone-lacZ strain was provided by Jeremy Nathans (Wang, Y. et al. (1992) Neuron 9, 429-40 (Johns Hopkins School of Medicine). All procedures involving animals were in compliance with the ARVO Statement for the Use of Animals in Ophthalmic and Vision Research.

Affymetrix array analysis: RNA was extracted as described previously (Punzo, C. & Cepko, C. (2007) Invest Ophthalmol Vis Sci 48, 849-57). Three to 4 retinae were used per extraction. A minimum of two arrays were analyzed per time point. The statistical significance of each gene expression profile was determined by a Jonckheere-Terpstra test of the hypothesized cone-death patterned alternative, using exact p-values calculated by the Harding algorithm (Harding, E. F. (1984) Applied Statistics 33, 1-6).

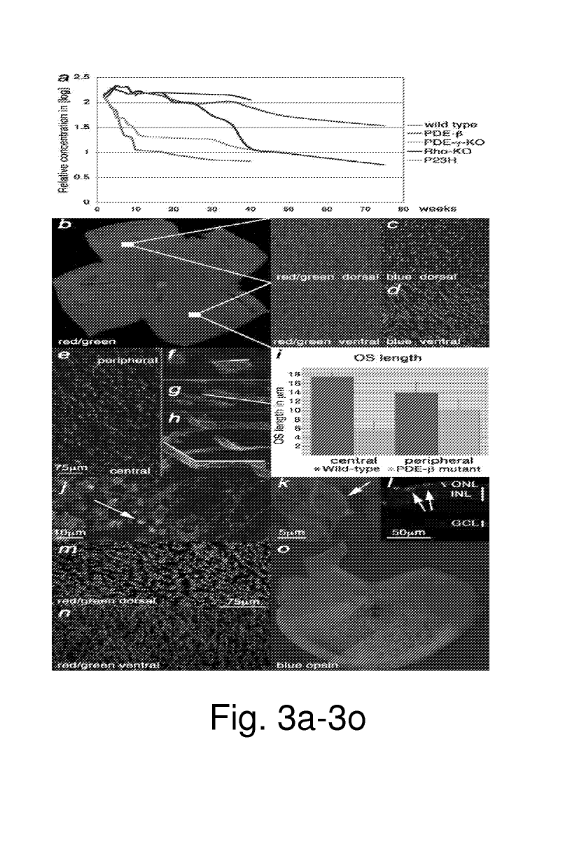

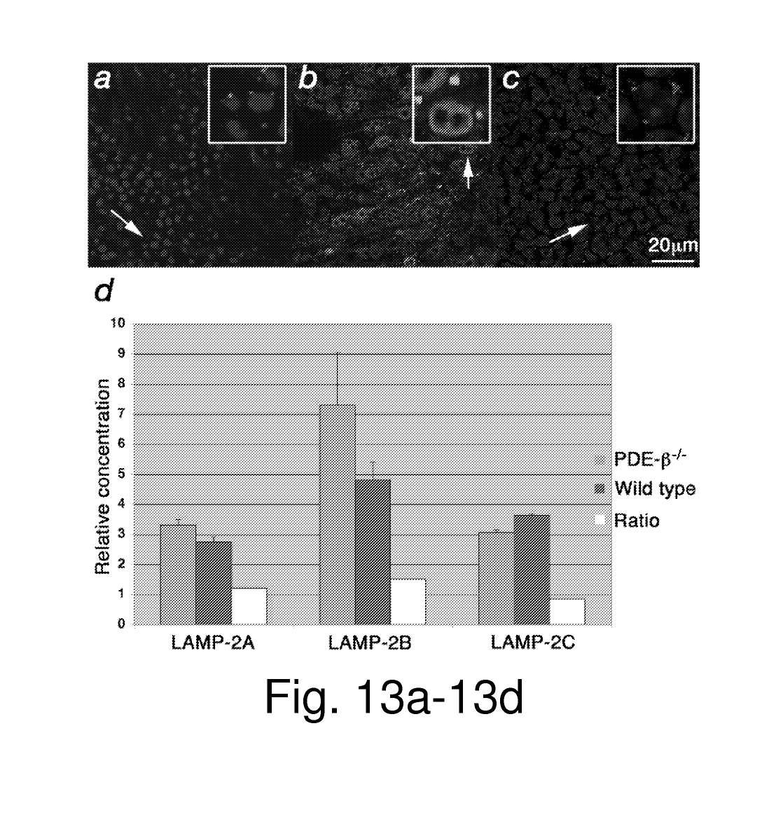

qRT-PCR was performed as described previously with the same primers and conditions for Opnlsw and gapdh (Punzo, C. & Cepko, C. (2007) Invest Ophthalmol Vis Sci 48, 849-57). The following primers and conditions were used for the three LAMP-2 splice forms: LAMP-2 forw. ctgaaggaagtgaatgtctacatg; LAMP-2A rev. gctcatatccagtatgatggc; LAMP-2B rev. cagagtctgatatccagcatag; LAMP-2C rev. gacagactgataaccagtacg. Conditions for all three PCRs: 95° for 3 sec, 52° for 15 sec, 72° for 25 sec. The data in FIG. 3a and FIG. 13d represent an average of 3 measurements corrected for gapdh.

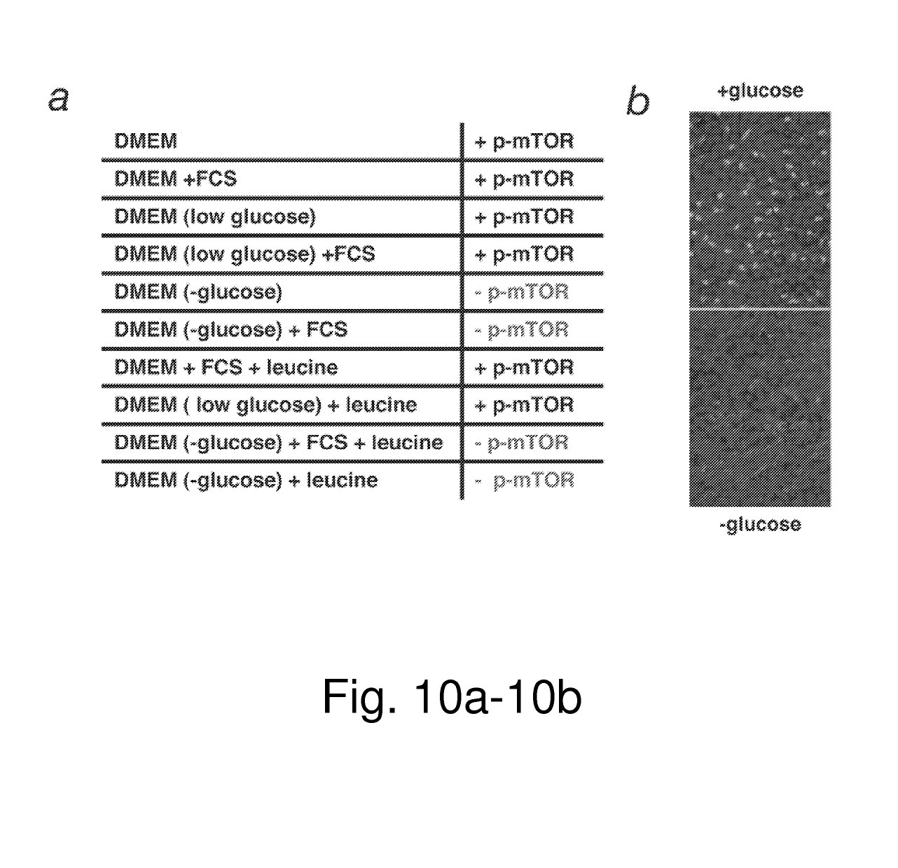

Retinal explant cultures: The retina was dissected free from other ocular tissues in DMEM, and then incubated in conditions according to the chart in FIG. 10a. Regular DMEM was at 4.5 g/L glucose, low glucose was at 1 g/L, leucine was added at 200 μM and FCS at 10%. Incubation was performed for 4 h and the retinae were fixed and processed for antibody staining as described below.

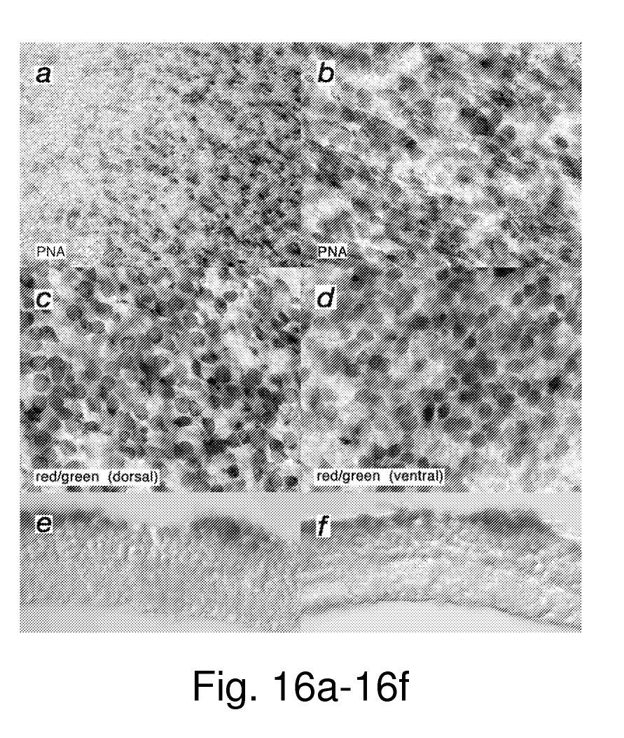

TUNEL, X-gal histochemistry and In Situ Hybridizations were performed as described previously (Punzo, C. & Cepko, C. (2007) Invest Ophthalmol Vis Sci 48, 849-57). For the double labeling of cones (see FIG. 16), retinae were first fixed in 2% PFA for 15 min. then processed for the X-GAL reaction and then post fixed in 4% PFA for 15 min. A biotin-PNA was used in an antibody staining procedure (see below) and detected with Streptavidin-POD (1:500, Roche) by a DAB stain (Sigma) according to the manufacture's instructions. The following ESTs were used for the red/green opsin and blue opsin probes respectively: red/green opsin (BE950633); blue opsin (BI202577). Probe for rhodopsin was generated by sub-cloning the coding sequence of the gene into pGEM-T Easy (Promega). The following primers were used for amplification of the coding sequence: forw. agccatgaacggcacagaggg (SEQ ID NO:1); rev. cttaggctggagccacctggct (SEQ ID NO:2). The antisense RNA was generated with T7 RNA polymerase.

Viral injections were performed as described previously (Punzo, C. & Cepko, C. L. (2008) Dev Dyn 237, 1034-42). Mice were injected at embryonic day 10 and harvested at postnatal week 10. The fusion protein was generated with a NotI site at the 5′ end followed by GFP, then LC3, and then an XhoI site at the 3′end and cloned into pQCXIX (Clonetech: cat. #631515). The following primers were used for the fusion protein: 5′NotI-GFP atgcgggccgccaccatggtgagcaagggcgaggagc (SEQ ID NO:3), 3′GFP-LC3 aggtcttctcggacggcatcttgtacagctcgtccatgccgag (SEQ ID NO:4), 5′LC3 atgccgtccgagaagaccttcaagc (SEQ ID NO:5), 3′LC3-XhoI atctcgagttacacagccattgctgtcccgaatg (SEQ ID NO:6).

Rapamycin, Streptozotocin and Insulin treatments were performed as follows. Rapamycin was diluted to 10 mg/ml in ethanol. The stock was diluted to 0.015 mg/ml in drinking water over a period of 2 weeks. A single intraperitoneal injection of 150 μl (12 mg/ml in 0.1M citric acid, ph4.5) of Streptozotocin was injected at postnatal day (P) 21. Insulin was injected intraperitoneally daily starting at P21. The concentration was increased weekly such that the first week, 10 U/kg body weight, the second 15 U/kg, the third 20 U/kg and fourth 30 U/kg body weight, were injected. In the treatment that lasted 7 weeks 30 U/kg body weight were injected for the remaining 3 weeks. Blood glucose levels were measured by collecting a drop of blood from the tail directly onto a test strip from TrueTrack smart system (CVS pharmacy). Eye bleeds were avoided due to the fact that cell survival in the retina was being assayed.

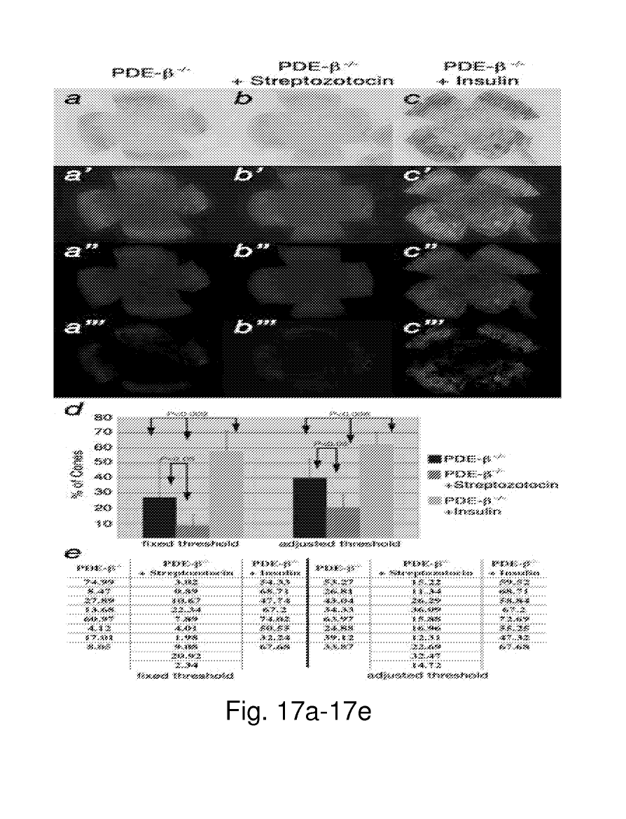

Quantification of cone survival was performed as follows. The colors of the bright light image were inverted and processed with Imaris software (Bitplane Inc) to calculate the percentage of blue surface area versus the total retinal surface area (see also FIG. 17). A minimum of 8 retinae per treatment, and for the control, were analyzed. P-values were calculated by the student's t-test. The cone lacZ transgene was chosen over PNA as a cone marker since the transgene labels cones more persistently, since, due to the shortening of the cone OS, PNA was found to stain less reliably than lacZ (see FIG. 16).

Whole mount and section antibody staining were performed as previously (Punzo, C. & Cepko, C. L. (2008) Dev Dyn 237, 1034-42) described with the following modifications. Antibody staining for LAMP-2: Triton was replaced with 0.01% Saponin. Antibody staining for p*-mTOR and p*-S6: PBS was replaced by TBS in every step of the procedure. Primary antibody dilutions: mouse a-rhodopsin Rho4D2 1:20051; goat α-β-Galactosidase (Serotec) 1:400; rabbit α-blue opsin (Jeremy Nathans) 1:1000; rabbit α-Gnat1 1:200 (Santa Cruz); rabbit α-Cleaved Caspase-3 (Cell Signaling) 1:100; rabbit α-Cleaved Lamin A (Cell Signaling) 1:100; rabbit α-GLUT-1 (Alpha Diagnostics) 1:100; rabbit α-p*-mTOR (Ser2448) (Cell Signaling) 1:300; rabbit α-p*-S6 (Ser235/236) (Cell Signaling) 1:100; rabbit α-HIF-1α (R&D Systems) 1:300; rat α-LAMP-2 (clone: GL2A7, from DSHB) 1:200. Time points analyzed for the rod and cone death kinetics (P: postnatal day; PW: postnatal week): PDE-β−/−: P10-P20 daily, PW3-10 weekly, PW 12, PW15, PW18, PW45; PDE-γ-KO: P10-P20 daily, PW3-PW10 weekly, PW15, PW25, PW45; Rho-KO: PW4-PW8 weekly, PW10, PW11, PW17, PW20, PW25, PW27, PW31, PW34, PW37, PW45, PW55, PW80; P23H: PW5, PW10, PW16, PW25, PW30, PW35, PW40, PW65, PW70, PW75, PW80, PW85.

### Example 1

### Rod and Cone Death Kinetics

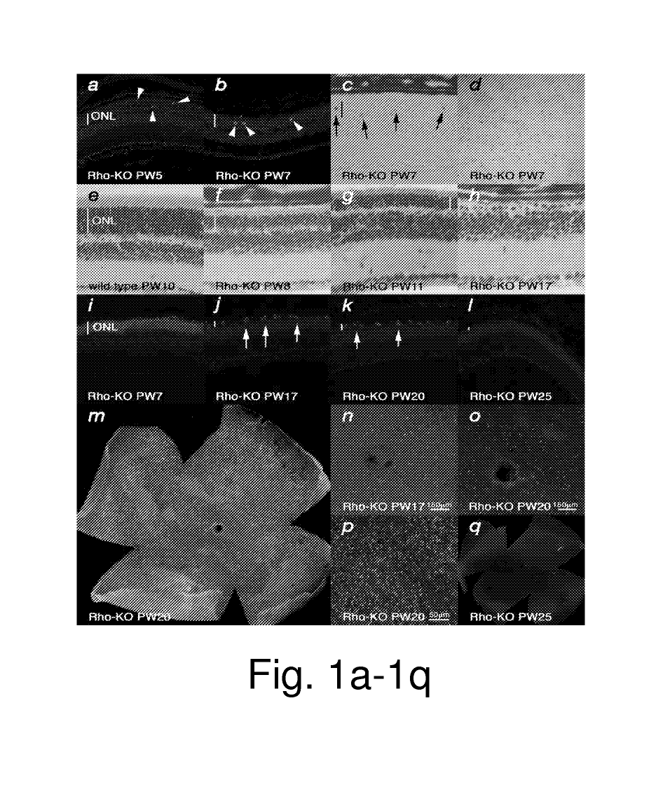

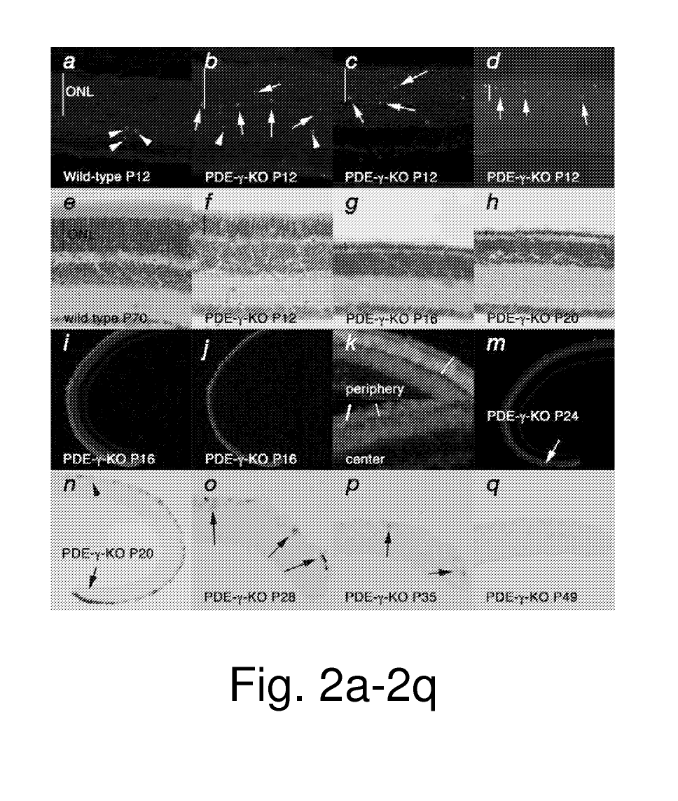

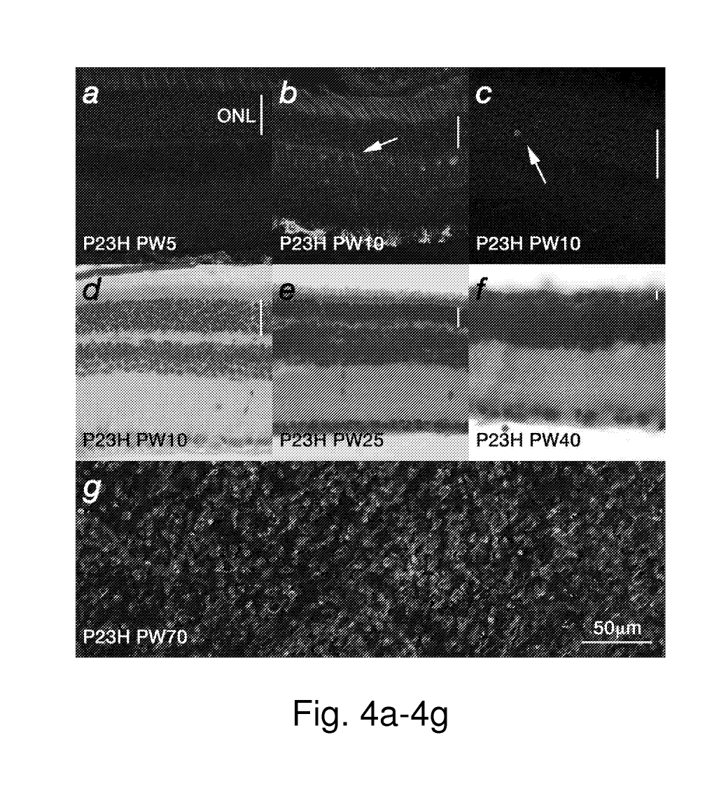

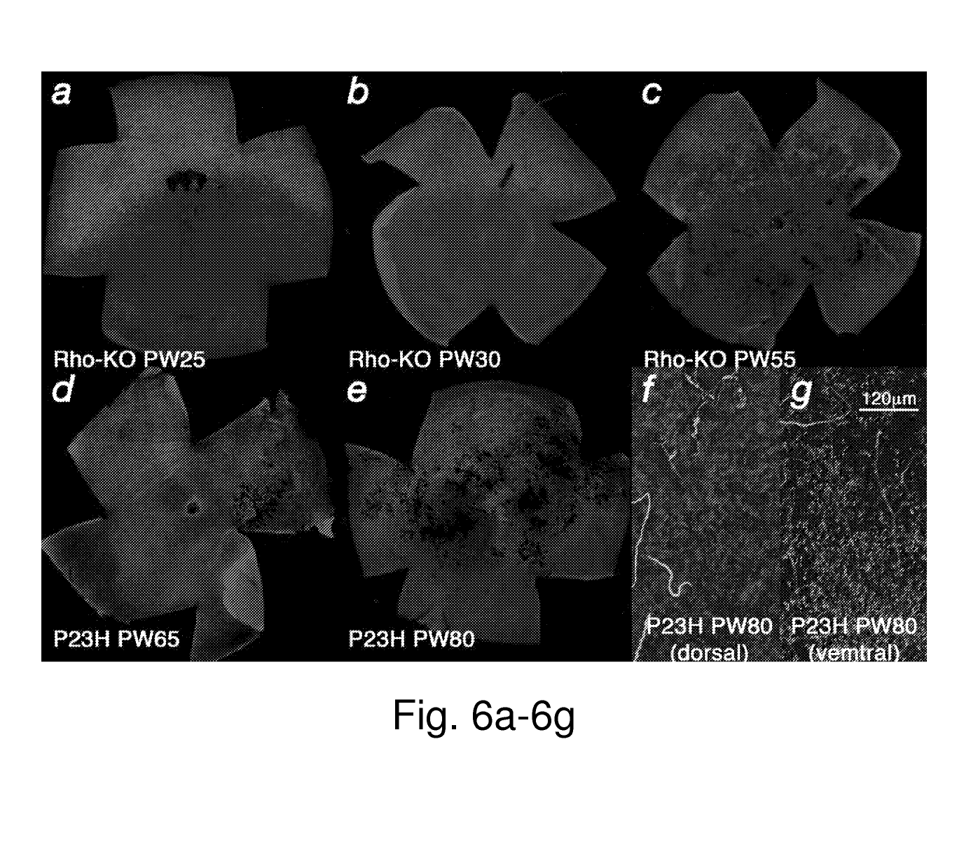

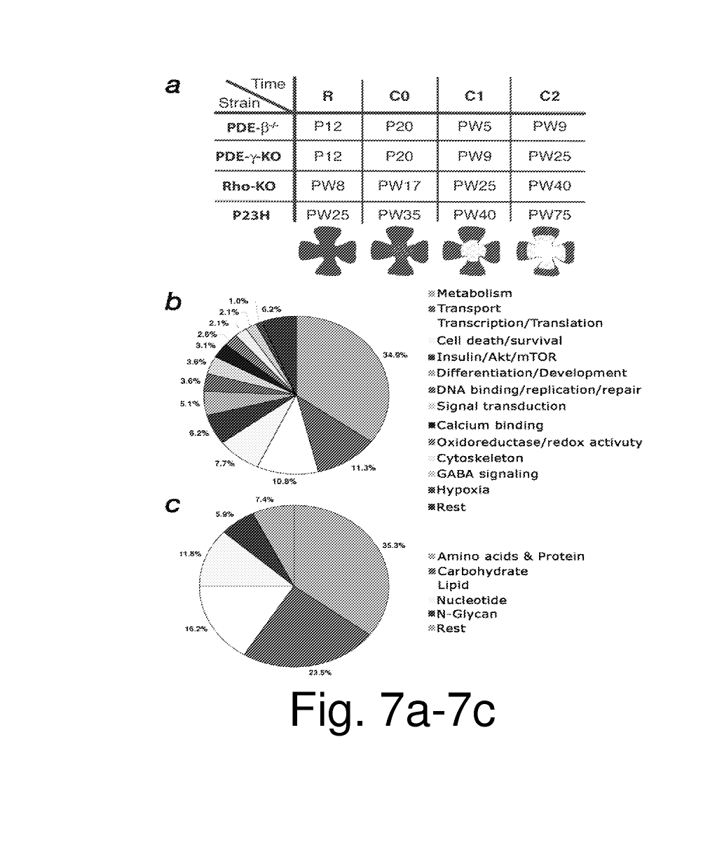

To establish a framework for comparing gene expression in 4 different models of RP, the equivalent stages of disease pathology were established through examination of the kinetics of rod (FIG. 1) (see also FIGS. 2 and 4) and cone (FIG. 3) (see also FIG. 6) death. Rod death kinetics were established by determining the onset, progression and end phase of rod death (FIG. 1). The time from the onset of rod death to the time when the outer nuclear layer (ONL) was reduced to 1 row of cells will be referred to as the major rod death phase. The time thereafter until rod death was complete will be referred to as the end phase of rod death. To determine the beginning of the major phase of rod death, cleavage of the nuclear envelope protein LaminA (FIG. 1a), and of the apoptotic protease Caspase3 (FIG. 1b), as well as TUNEL (FIG. 1c, d) were used. The continuation of the major rod death phase was monitored by these assays, as well as inspection of histological sections (FIG. 1e-h), as rods account for more than 95% of all PRs. Once the ONL reached one row of cells, the major phase of rod death was over. The end phase of rod death was determined using rod-specific markers to perform either in situ hybridization (see FIG. 2) or immunohistochemistry (FIG. 1i-l) on retinal sections. However, unless every section of a single retina is collected it is difficult to determine if any rods remain. Thus, retinal flat mounts also were used to allow a comprehensive analysis of the end phase of rod death (FIG. 1m-q). Interestingly, while in the two PDE mutants and in the Rho-KO mutant the end phase of rod death was clearly defined, in the P23H mutant, rods died so slowly that even 50 weeks (latest time point analyzed) after the end of the major phase of rod death, some rods were still present (see FIG. 4).

Two methods were used to determine the onset and progression of cone death. First, the overall time frame of cone demise was determined by quantitative real-time polymerase chain reaction (qRT-PCR) (FIG. 3a) for the ventral (Applebury, M. L. et al. (2000) Neuron 27, 513-23) cone specific transcript Opnlsw (opsin1 short-wave-sensitive: blue cone opsin). This allowed for an initial quantitative comparison among different strains, but was not adequate to determine the number of cones as transcript levels could vary prior to cell death. Next, whole mount immunohistochemistry for red/green opsin (Opnlmw: opsin1 medium-wave-sensitive) and peanut agglutinin lectin (PNA) were used (FIG. 3b-n). Both markers are expressed throughout the murine retina allowing for the visualization of cones (FIG. 3b-d). Interestingly, the onset of cone death always occurred at the equivalent stage of rod death, namely after the major rod death phase, when the thickness of the ONL was reduced to only a single row of cells. Cone death was found to proceed from the center to the periphery in all 4 models, as seen by staining with PNA (FIG. 3e). It was preceded by a gradual reduction of the outer segment (OS) length (FIG. 3f-i) and by opsin localization from the OS to the entire cell membrane (FIG. 3j-l). In addition, red/green opsin (Opnlmw) protein, which is normally detected throughout the mouse retina (FIG. 3b), was detected mainly dorsally during cone degeneration (FIG. 3m, n). However, PNA staining showed no appreciable difference across the dorsal/ventral axis (FIG. 3m, n). Similarly, blue opsin expression, which is normally detected only ventrally (Applebury, M. L. et al. (2000) Neuron 27, 513-23) (FIG. 3c, d), was not affected during degeneration (FIG. 3o). Shortening of cone OSs and loss of cone-specific markers has also been described in human cases of RP (John, S. K., et al. (2000) Mol Vis 6, 204-15).

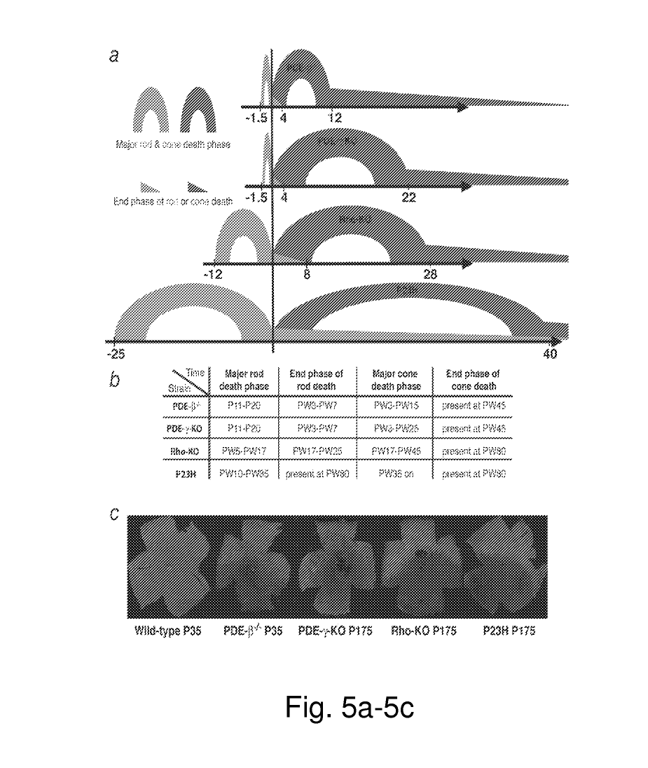

In summary, the kinetics and histological changes that accompanied rod and cone death shared several features across the 4 models. First, cone degeneration always started after the major rod death phase (FIG. 5a, b). This point was reached at very different ages in three of the 4 mutants, as the overall kinetics of rod death were quite different. Second, cone death was always central to peripheral and was preceded by a reduction in OS length. Third, in all 4 mutants, red/green opsin protein levels were detectable mainly dorsally during cone degeneration (FIG. 5c). These common features evidence a common mechanism(s) of cone death. Moreover, gene expression changes that were common across the 4 models at the onset of cone death serve to elucidate this common mechanism.

### Example 2

### Microarray Analysis

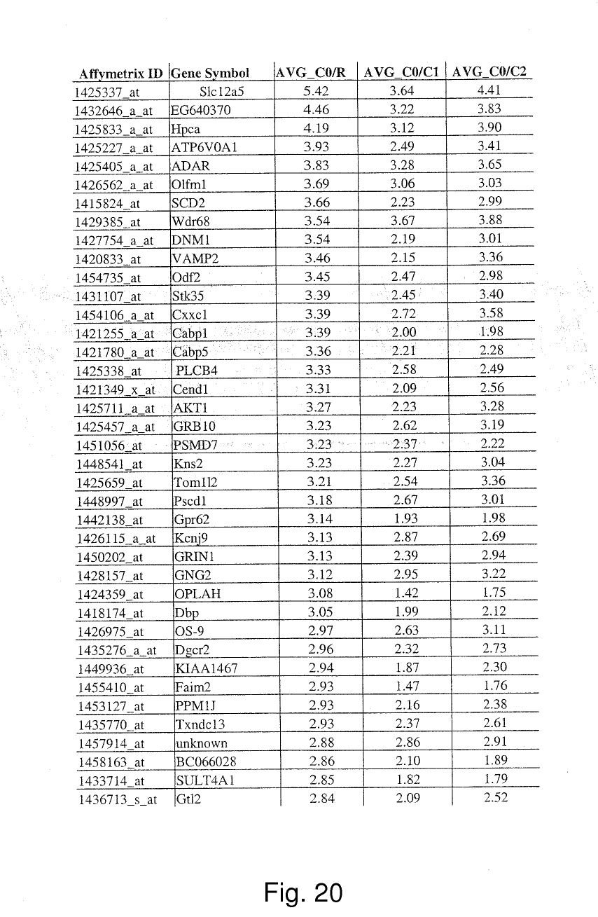

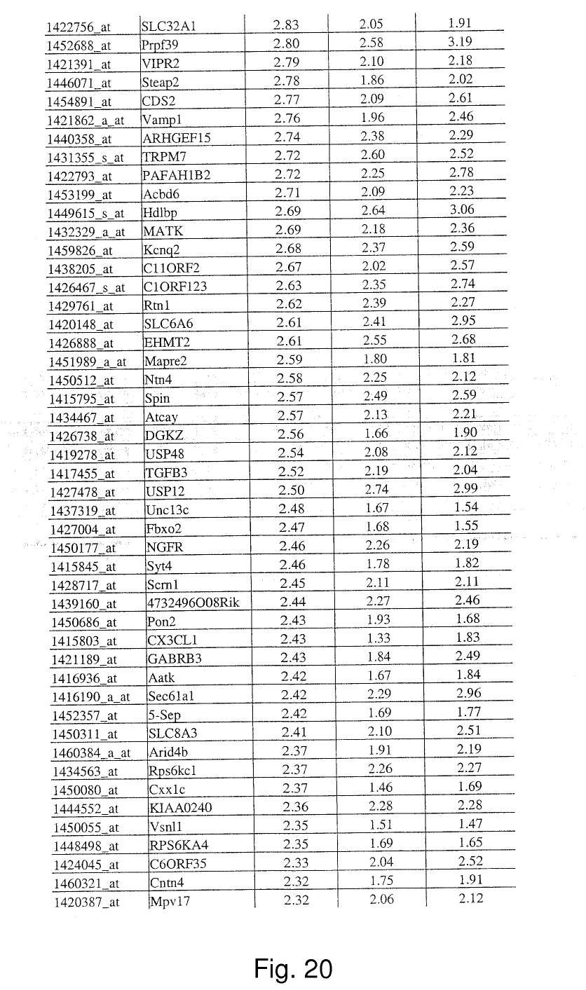

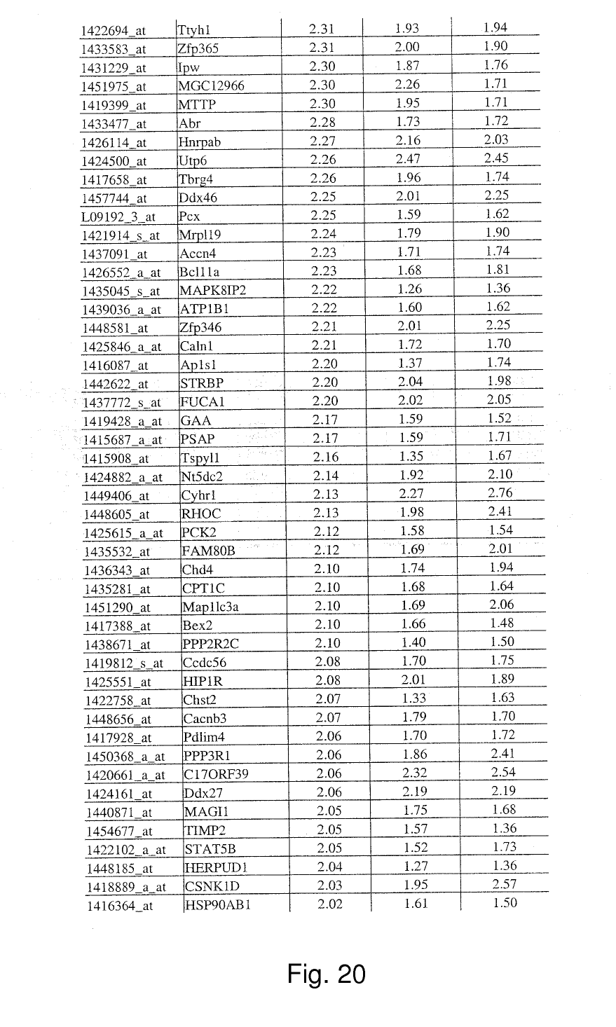

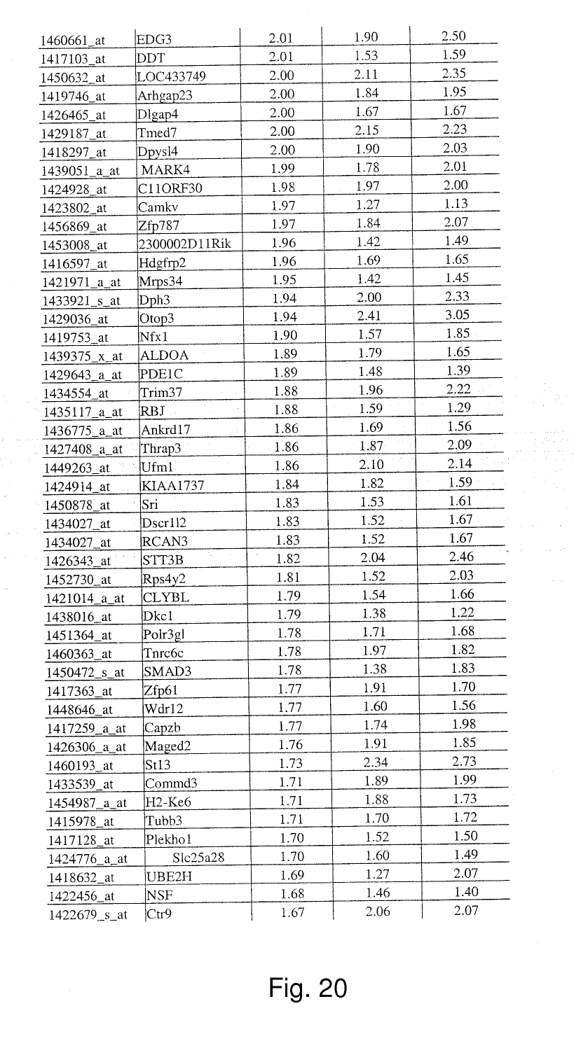

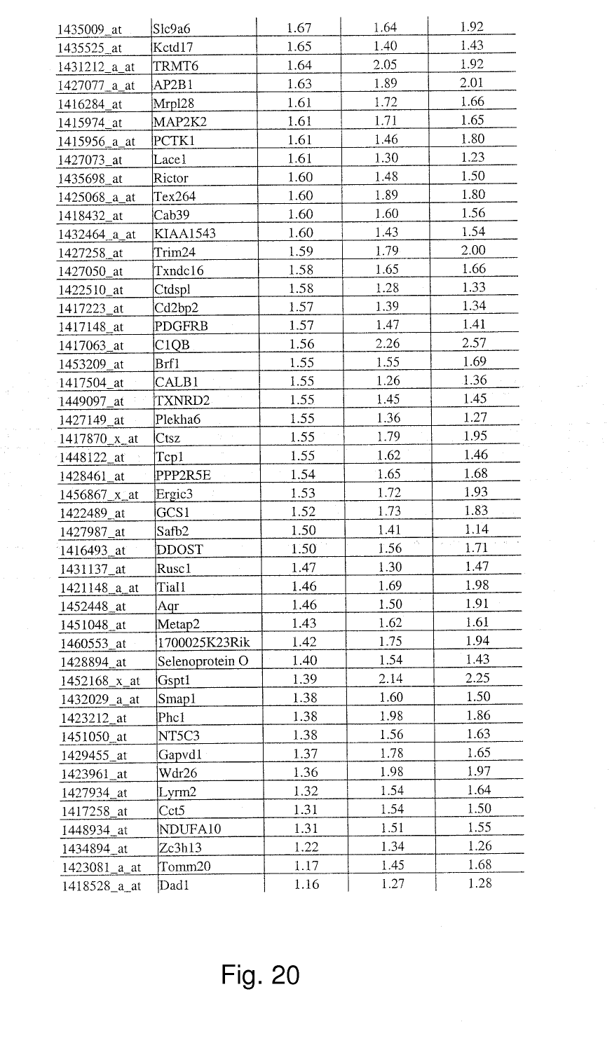

To determine common gene expression changes, RNA samples from all 4 models were collected halfway through the major phase of rod death, at the onset of cone death, and from two time points during the cone death phase (FIG. 7a). The RNA was then hybridized to an Affymetrix 430 2.0 mouse array. Gene expression changes were compared within the same strain across the 4 time points. Two criteria had to be fulfilled to select a gene for cross comparison among the 4 strains. First, the change over time had to be statistically significant (see Material & Methods). Second, a gene had to be upregulated at least 2 fold at the onset of cone death compared to the other three time points. This second criterion removed rod-specific changes that were still occurring at the onset of cone death while at the same time enriched for changes at the onset of cone death. A total of 240 Affymetrix IDs were found that satisfied both criteria within each of the 4 strains. The 240 IDs matched to 230 genes (see FIG. 20). Of the 195 genes that could be annotated, 34.9% (68 genes) were genes involved in cellular metabolism (FIG. 7b, c). The signaling pathway with the highest number of hits (12 genes) was the insulin/mTOR (mammalian target of rapamycin) signaling pathway (FIG. 7b), a key pathway in regulating many aspects of cellular metabolism. Thus, the data evidences that events at the onset of cone death coincided with changes in cellular metabolism likely to be regulated by the insulin/mTOR pathway.

### Example 3

### mTOR in Wild Type and Degenerating Retinae

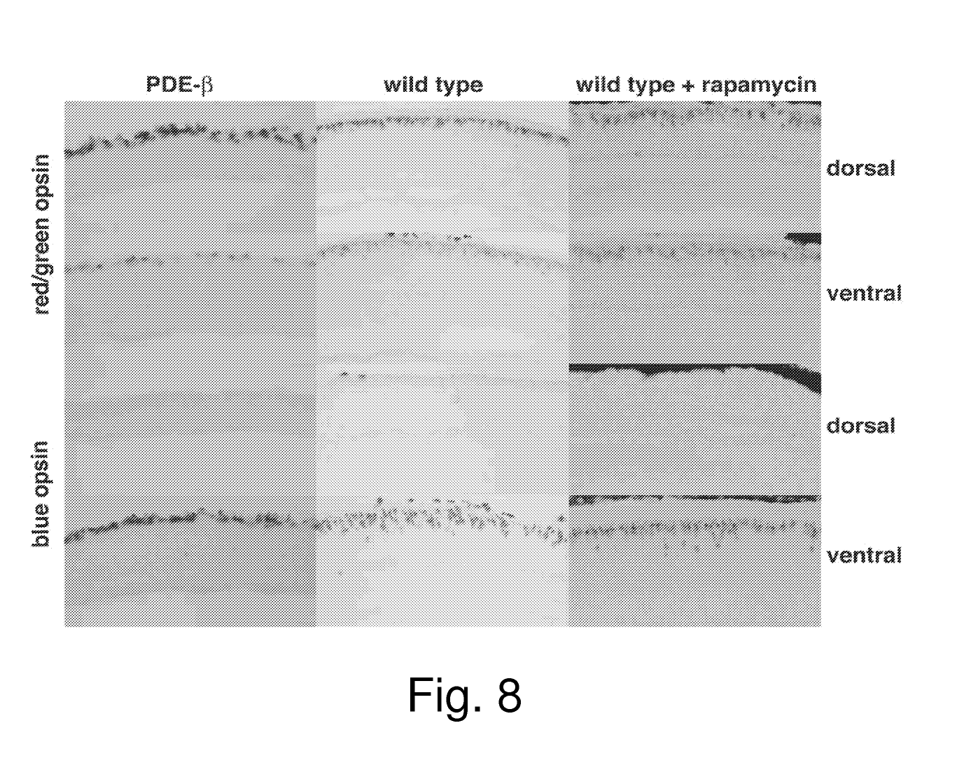

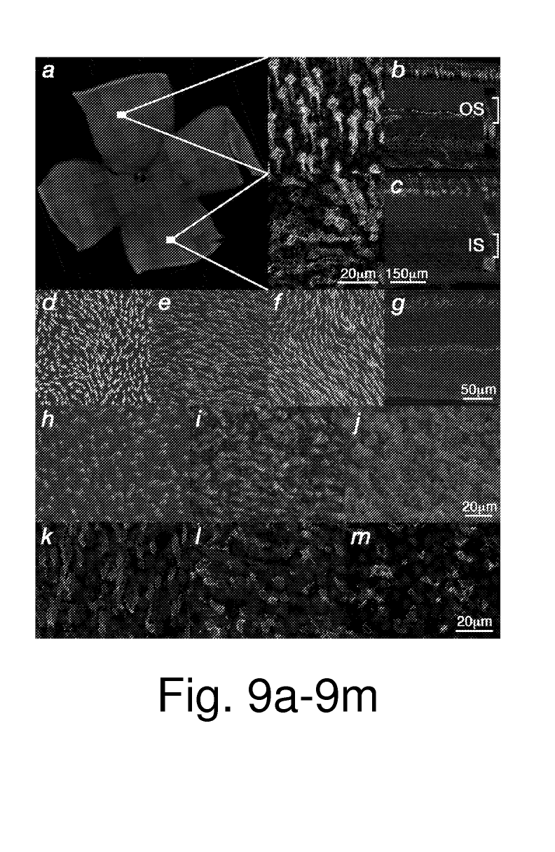

Based on the findings of the microarray analysis, the insulin/mTOR signaling pathway was examined during the period of cone death. The kinase, mTOR, is a key regulator of protein synthesis and ribosome biogenesis (Reiling, J. H. & Sabatini, D. M. (2006) Oncogene 25, 6373-83). When cellular energy levels are high, mTOR allows energy consuming processes, such as translation, and prevents autophagy, while nutrient poor conditions have the reverse effect. Therefore, glucose, which increases cellular ATP levels, and amino acid availability, especially that of leucine, positively affect mTOR activity. To understand if cellular energy levels or amino acid availability might be compromised in cones during degeneration, levels of phosphorylated mTOR (p*-mTOR) were examined by immunofluorescence. Phosphorylation of mTOR increases kinase activity, and therefore levels of p*-mTOR can serve as an indicator of its activity level. Since every eukaryotic cell expresses mTOR, a certain level of p*-mTOR is likely to be found in every cell. Surprisingly, high levels of p*-mTOR were detected only in dorsal cones of wild type retinae (FIG. 9a-c). This phosphorylation pattern was reminiscent of the red/green opsin pattern seen during cone degeneration (FIG. 5c). Since mTOR is a key regulator of translation, we investigated whether the ventral red/green opsin downregulation that occurred during cone degeneration could be mimicked by a reduction in mTOR activity. To this end, wild type mice were treated with rapamycin, an mTOR inhibitor18. This treatment resulted in ventral downregulation of red/green opsin, without affecting blue opsin or PNA staining or the dorsal phosphorylation of mTOR itself (FIG. 9d-g). Thus, inhibition of mTOR in wild type recapitulated the expression of red/green opsin and blue opsin, as well as the pattern of PNA staining, in the mutants during degeneration, indicating that the ventral downregulation of red/green opsin seen during degeneration might be due to reduced mTOR activity. As expected for mTOR function, the downregulation of red/green opsin did not occur at the RNA level, but at the protein level, in untreated mutant mice, as well as in wild type mice treated with rapamycin (see FIG. 8). Finally, analysis of mutant retinae showed a decrease of p*-mTOR levels in dorsal cones during cone degeneration (FIG. 9h-m). To test whether the high level of p*-mTOR found in dorsal wild type cones was glucose-dependent, retinal explants of wild type mice were cultured in media for 4 hours in the presence or absence of glucose. Dorsal p*-mTOR was abolished in the absence of glucose even when leucine concentrations were increased in the medium (see FIG. 10). Thus, the data on mTOR establish a link between mTOR activity, the expression changes of red/green opsin seen during degeneration, and the microarray data, which indicated metabolic changes at the onset of cone death. Those changes may be caused by compromised glucose uptake in cones.

### Example 4

### Responses of Cones to Nutritional Imbalance

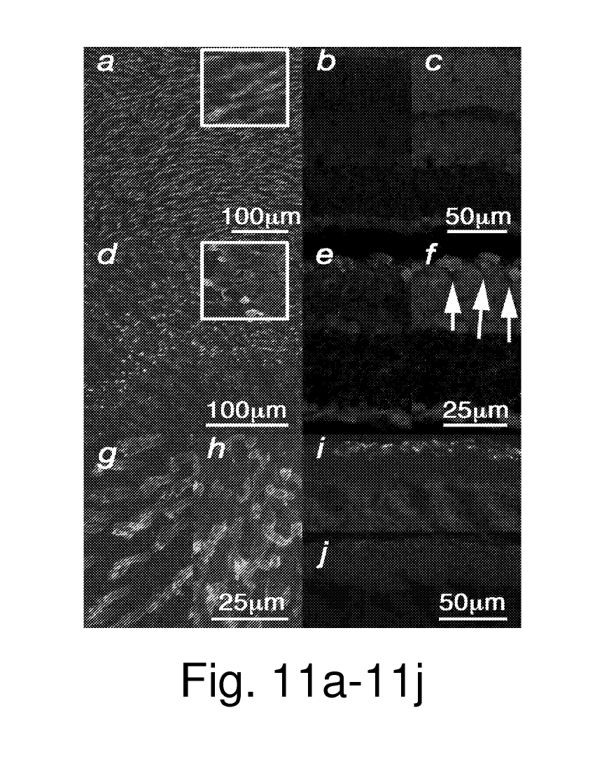

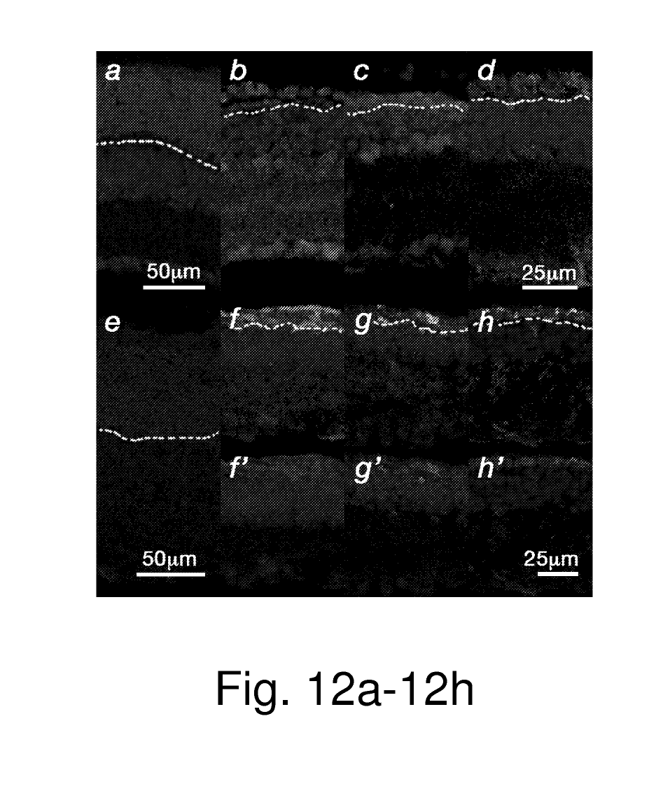

The data on mTOR evidenced a nutritional imbalance in cones during cone degeneration, possibly caused by reduced glucose levels in cones. To test this idea, the level of the heterodimeric transcription factor, Hypoxia inducible factor 1 (HIF-1α/β), which improves glycolysis under stress conditions such as low oxygen, was examined. HIF-1 and mTOR are tightly linked as low oxygen results in low energy due to reduced oxidative phosphorylation, and therefore in reduced mTOR activity (Reiling, J. H. & Sabatini, D. M. (2006) Oncogene 25, 6373-83; Dekanty, A., et al. (2005) J Cell Sci 118, 5431-41; Hudson, C. C. et al. (2002) Mol Cell Biol 22, 7004-14; Treins, C., et al. (2002) J Biol Chem 277, 27975-81; Zhong, H. et al. (2000) Cancer Res 60, 1541-5; Thomas, G. V. et al. (2006) Nat Med 12, 122-7). An upregulation of the regulated subunit HIF-1α would likely reflect low glucose levels in cones, and not hypoxic conditions, as oxygen levels are increased due to the loss of rods (Yu, D. Y. & Cringle, S. J. (2005) Exp Eye Res 80, 745-51). Immunofluorescence analysis of HIF-1α during cone degeneration revealed an upregulation of the protein in cones in all 4 mouse models (FIG. 11a-f and 12a-d). Consistent with the upregulation of HIF-1α, glucose transporter 1 (GLUT1), a HIF-1α target gene (Wang, G. L., et al. (1995) Proc Natl Acad Sci USA 92, 5510-4; Ebert, B. L., et al. (1995) J Biol Chem 270, 29083-9) also was found to be upregulated in cones, again in all 4 mouse models (FIG. 11g-j and FIG. 12e-h). Thus HIF-1α and GLUT1 upregulation are consistent with a response in cones to overcome a shortage of glucose. It also provides a link to the decreased p*-mTOR levels found during degeneration as well as the sensitivity of p*-mTOR to glucose.

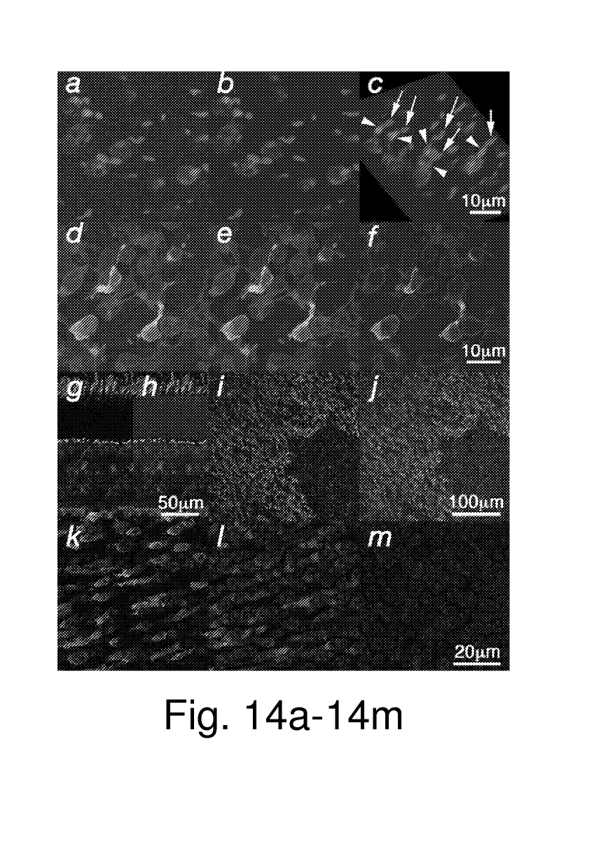

To ascertain if cones are nutritionally deprived, autophagy within cones was assessed. Two types of autophagy are inducible by various degrees of nutrient deprivation: macroautophagy and chaperone mediated autophagy (CMA) (Massey, A., et al. (2004) Int J Biochem Cell Biol 36, 2420-34; Finn, P. F. & Dice, J. F. (2006) Nutrition 22, 830-44; Codogno, P. & Meijer, A. J. (2005) Cell Death Differ 12 Suppl 2, 1509-18; Dice, J. F. (2007) Autophagy 3, 295-9). Macroautophagy is non-selective, targets proteins or entire organelles, and is marked by de novo formation of membranes that form intermediate vesicles (autophagosomes) that fuse with the lysosomes. The machinery required for macroautophagy has been shown to be present in PRs (Kunchithapautham, K. & Rohrer, B. (2007) Autophagy 3, 433-41). In contrast, CMA is selective and targets individual proteins for transport to the lysosomes. The presence of macroautophagy was assessed by infection with a viral vector encoding a fusion protein of green fluorescent protein (GFP) and light chain 3 (LC3), an autophagosomal membrane marker (Kabeya, Y. et al. (2000) Embo J 19, 5720-8; Mizushima, N., et al. (2004) Mol Biol Cell 15, 1101-11; Punzo, C. & Cepko, C. L. (2008) Dev Dyn 237, 1034-42). No difference was observed in GFP distribution in cones of wild type and mutant mice, indicating that formation of autophagosomes was absent during cone death (see FIG. 14a-f). Additionally, high levels of phosphorylated ribosomal protein S6 were found in all, or most, cones (see FIG. 14g-h) reflecting an increased activity of ribosomal S6 kinase 1 (S6K1), an inhibitor of macroautophagy (Codogno, P. & Meijer, A. J. (2005) Cell Death Differ 12 Suppl 2, 1509-18). Consistent with these findings is the fact that macroautophagy reflects an acute short-term response to nutrient deprivation or cellular stress conditions (Massey, A., et al. (2004) Int J Biochem Cell Biol 36, 2420-34; Finn, P. F. & Dice, J. F. (2006) Nutrition 22, 830-44). Prolonged non-selective degradation of newly synthesized proteins to overcome the stress condition would not be favorable to cells and would likely result in the relatively rapid death of most cones, rather than the slow death seen in RP.

CMA is normally activated over extended periods of starvation and results in increased levels of lysosomal-associated membrane protein (LAMP) type 2A at the lysosomal membrane (Massey, A., et al. (2004) Int J Biochem Cell Biol 36, 2420-34; Finn, P. F. & Dice, J. F. (2006) Nutrition 22, 830-44; Cuervo, A. M. & Dice, J. F. (2000) Traffic 1, 570-83). Both starvation and oxidative stress can induce CMA Massey, A., et al. (2004) Int J Biochem Cell Biol 36, 2420-34). Starvation increases LAMP-2A by preventing its degradation while oxidative stress results in de novo synthesis of LAMP-2A (Kiffin, R., et al. (2004) Mol Biol Cell 15, 4829-40). A LAMP-2 antibody that recognizes the proteins resulting from all 3 splice isoforms (Cuervo, A. M. & Dice, J. F. (2000) J Cell Sci 113 Pt 24, 4441-50) (A, B, C) showed high levels of LAMP-2 at the lysosomal membrane in all 4 mutants during cone degeneration (FIG. 13a-c; data only shown for PDE-β−/−). The high levels were specific to cones and were not seen in cells of the inner nuclear layer (FIGS. 13b, c), which might reflect the possibility that cones are the only starving cells in the RP retina. qRT-PCR for the three splice isoforms showed only a minor increase in mRNA levels of LAMP-2A (1.2×) and a decrease in LAMP-2C (FIG. 13d) indicating that the increase seen in protein at the membrane is mainly due to nutritional deprivation and only to a lesser extent to oxidative stress (Komeima, K., et al. (2006) Proc Natl Acad Sci USA 103, 11300-5; Komeima, K., et al. (2007) J Cell Physiol. 275, 28139-28143; Kiffin, R., et al. (2004) Mol Biol Cell 15, 4829-40). Taken together, the data demonstrates that nutritional imbalance in cones leads to the activation of CMA, a process that is consistent with prolonged starvation.

### Example 5

### Stimulation of the Insulin Receptor Pathway Prolongs Cone Survival

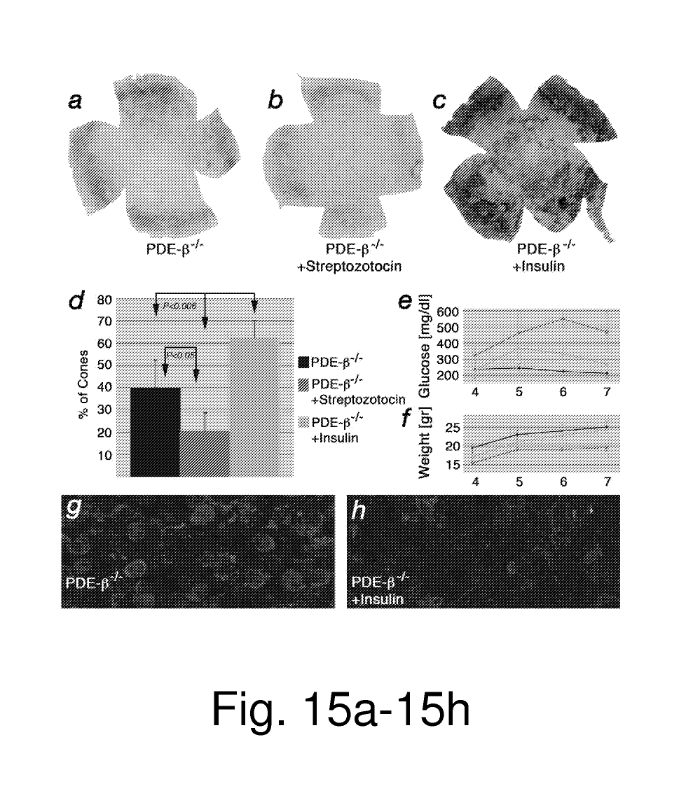

The data on mTOR, HIF-1α, GLUT1 and the induction of CMA demonstrated that a shortage of glucose in cones resulting in starvation and further demonstrated that the insulin/mTOR pathway plays an important role during cone death. To determine if the insulin/mTOR pathway can influence cone survival, we stimulated the pathway by systemic treatment of PDE-β−/− mice with insulin. The PDE-β mutant was chosen over the other three mutants due to its faster cone death kinetics, allowing for a better read-out of cone survival. Mice were treated with daily intraperitoneal injections of insulin over a 4 week period, starting at the onset of cone death. To reduce insulin, a single injection of streptozotocin, a drug that kills the insulin-producing beta cells of the pancreas, also was examined. Systemic administration of insulin results in a desensitized insulin receptor due to a feedback loop in the pathway, which causes an increase in blood glucose levels. Injection of streptozotocin, which also results in increased blood glucose levels, served as a control for the effect of elevated blood glucose, and also provided animals with reduced levels of insulin. PDE-β−/− mice injected with insulin showed improved cone survival compared to uninjected control mice. PDE-β−/− mice injected with Streptozotocin showed a decrease in cone survival (FIG. 15a-d). Improved cone survival was therefore due to insulin and not to the increased blood glucose levels (FIG. 15e). Additionally, cones in mutant mice treated with insulin did not show the upregulation of HIF-1α seen normally in cones during degeneration, consistent with the notion that cones were responding to insulin directly (FIGS. 15g, h).

### Discussion

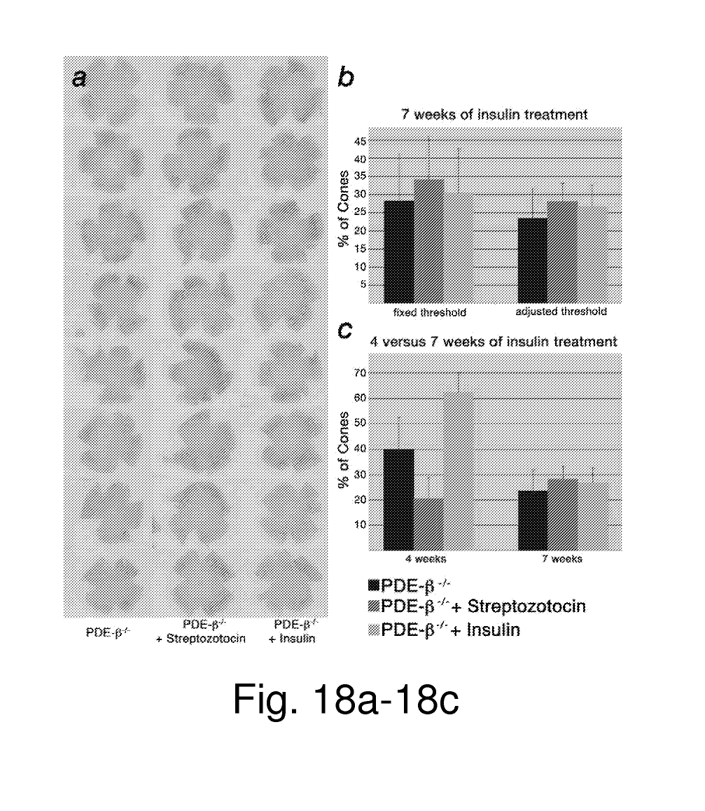

The results presented herein show that cones exhibit signs of nutritional imbalance during the period of cone degeneration in RP mice. The microarray analysis demonstrates that there are changes in cellular metabolism involving the insulin/mTOR pathway at the onset of cone death. It was demonstrated that inhibition of mTOR in wild type mice resulted in the same pattern of loss of red/green opsin as seen during degeneration. In accord with changes in p*-mTOR, and its sensitivity to glucose, an upregulation of HIF-1α and GLUT1 was observed, demonstrating that glucose uptake, and/or the intracellular levels of glucose, may be compromised in cones of RP mice. Additionally, systemic administration of insulin prolonged cone survival, whereas depletion of endogenous insulin had the reverse effect. The systemic treatment with insulin prevented the upregulation of HIF-1α in cones seen normally during cone degeneration, demonstrating that insulin was directly acting on cones. Interestingly, a prolonged treatment of insulin during a time span of 7 weeks instead of 4 weeks did not show any significant improvement of cone survival (see FIG. 18). This may reflect the feedback loop of the pathway in which S6K1 acts directly onto the insulin-receptor substrate (IRS). The results indicate that nutrient availability in cones may be altered during the period of cone degeneration and that the insulin/mTOR pathway plays a crucial role. A recent report showed that constitutive expression of proinsulin in the rd10 mouse model of RP delays photoreceptor death, both of rods and cones (Corrochano, S. et al. (2008) Invest Ophthalmol Vis Sci 49, 4188-94). However, proinsulin seems not to act through the insulin receptor as mice treated with proinsulin did not develop hyperglycemia. Proinsulin blocks developmental cell death and thus may interfere with the apoptotic pathway in the postnatal retina. Macroautophagy, which is controlled by mTOR through its downstream target S6K1, was not detected during cone degeneration, while CMA appeared to be activated. Increased LAMP-2A levels at the lysosomal membrane indicated activation of CMA. In addition, the observations concerning mTOR, HIF-1α, and GLUT1 are consistent with starvation and CMA. The lack of detectable macroautophagy does not rule out the possibility that macroautophagy might occur for a short period of time (e.g., 24 hours) prior to the activation of CMA. The data only show that macroautophagy is not the main form of autophagy over an extended period of time, which is consistent with the notion that macroautophagy is a short-term response. The prolonged inhibition of macroautophagy is likely due to increased S6K1 activity as seen by increased p*-S6 levels. S6K1 is positively regulated by mTOR and AMP-activated protein kinase (AMPK) (Codogno, P. & Meijer, A. J. (2005) Cell Death Differ 12 Suppl 2, 1509-18), which reads out cellular ATP levels. Therefore, while mTOR may report metabolic problems with respect to glucose uptake, and reduce energy consuming processes and improve glycolysis through HIF-1α, AMPK may report normal cellular ATP levels and inhibit macroautophagy. This represents a specific response to the energy requirements of cones. Most of the glucose taken up by PRs never enters the Krebs cycle (Poitry-Yamate, C. L., et al. (1995) J Neurosci 15, 5179-91). Thus the shortage of glucose may not cause a shortage of ATP. Lactate, provided by Muller glia, can generate ATP via the Krebs cycle (Poitry-Yamate, C. L., et al. (1995) J Neurosci 15, 5179-91). However, glucose is needed to generate NADPH in the pentose phosphate cycle, and NADPH is required for synthesis of phospholipids, the building blocks of cell membranes. PRs constantly shed their membranes at the tip of the OS s. Since reduced levels of glucose would result in reduction of membrane synthesis, the rate of OS phagocytosis by the RPE may be higher than the rate of membrane synthesis by cones. Consistent with this, OS shortening preceded cell death in these 4 models, as is also observed in human cases of RP17. Additionally, changes that affect lipid metabolism were also seen by the microarray analysis.

These studies described herein were designed to determine why the loss of rods result in cone death in RP. The previous hypotheses attributing cone death either to a toxin released by rod cells or to the lack of a trophic factor produced by rod cells and necessary for cone survival each fail to explain the pathology found in humans. The rod and cone death kinetics shown here clearly argue against a toxin produced by dying rods as a cause for cone death since the onset of cone death always occurred after the major rod death period. If a rod toxin caused cone death, then the onset of cone death should have either coincided with the onset of rod death or should have started shortly thereafter, since this would be the period of peak toxin production. Interestingly, the lack of a trophic factor produced by healthy rods and required for cone survival would agree with the onset of cone death seen in all four models as one would expect the onset of cone death during the end stages of rod death. However, the progression of cone death and the end phase of rod death make this unlikely hypothesis as the sole reason for cone death. In the two PDE mutants and in the Rho-KO mutant, cones were dying for many weeks after the end phase of rod death, indicating that they could survive quite awhile in the absence of rods. In addition, in the P23H model, rods died so slowly during the end phase of rod death, that during the entire period of cone death, rods were still present. The hypothesis that a lack of a rod trophic factor being the main cause for cone death seems unlikely given these discrepancies.

The observations described herein of nutritionally deprived cones demonstrate the dependence of cones on rods. The OS-RPE interactions are vital since the RPE shuttles nutrition and oxygen from the choroidal vasculature to PRs. Roughly 95% of all PRs in mouse and human are rods and approximately 20-30 OSs contact one RPE cell (Snodderly, D. M., et al. (2002) Invest Ophthalmol Vis Sci 43, 2815-8; Young, R. W. (1971) Journal of Cell Biology 49, 303-318). Thus, only 1-2 of those RPE-OS contacts are via cones. During the collapse of the ONL, the remaining cone:RPE interactions are likely perturbed. If these interactions drop below a threshold required for the proper flow of nutrients, the loss of rods results in a reduced flow of nutrients to cones. In all 4 mouse models, the onset of cone death occurred when the ONL reached one row of cells. This cell density therefore represents the critical threshold. Then, while the remaining rods die due to a mutation in a rod-specific gene, cone death begins due to nutrient deprivation. In accord with this notion, cone death progressed more slowly when the remaining rods died slowly. This mechanism would also explain why the loss of cones does not lead to rod death (Biel, M. et al. (1999) Proc Natl Acad Sci USA 96, 7553-7; Yang, R. B. et al. (1999) J Neurosci 19, 5889-97). Since in humans and mouse, cones are less than 5% of all PRs, the critical threshold that perturbs OS-RPE interactions would not be reached. Further support for this idea is provided by studies in zebrafish where the overall ratio of rods to cones is reversed (1:8). Additionally, the distribution of rods and cones in zebrafish is uneven such that certain regions are cone-rich whereas other regions are rod-rich. A recently isolated mutation in a cone-specific gene resulted in rod death, but only in regions of high cone density (Stearns, G., et al. (2007) J Neurosci 27, 13866-74), leading Stearns and co-workers to conclude that cell density is the crucial determinant. We determined that once a critical threshold of cell density is breached, improper OS-RPE interactions result in reduced flow of nutrients (e.g., glucose). This results in reduced OS membrane synthesis, which in turn further contributes to a reduced uptake of nutrients from the RPE. Ultimately, prolonged starvation, as indicated by the activation of CMA, leads to cell death. Since starvation can occur slowly over extended periods of time, and because the rate may fluctuate due to fluctuations in nutrient uptake, the slow and irregular demise of cones observed in humans results therefrom. Therefore, the results presented herein not only provide a new mechanism of cone death in RP that should direct future therapeutic approaches, but also consolidate the data from the literature with respect to the death kinetics of rods and cones seen in mice and patients with different RP mutations.

### Equivalents

Those skilled in the art will recognize, or be able to ascertain using no more than routine experimentation, many equivalents to the specific embodiments of the invention described herein. Such equivalents are intended to be encompassed by the following claims.

1. A method for treating or preventing a retinal disorder in a subject comprising administering to said subject an mTOR modulator in an amount effective for modulating mTOR activity in the subject, thereby treating or preventing the retinal disorder in the subject.

2. The method of claim 1, wherein the retinal disorder is retinitis pigmentosa.

3. The method of claim 1, wherein the retinal disorder is associated with decreased viability of cone cells.

4. The method of claim 1, wherein the retinal disorder is associated with decreased viability of rod cells.

5. The method of claim 1, wherein the retinal disorder is a genetic disorder.

6. The method of claim 1, wherein the retinal disorder is not diabetic retinopathy.

7. The method of claim 1, wherein the retinal disorder is not associated with blood vessel leakage and/or growth.

8. A method for treating or preventing retinitis pigmentosa in a subject comprising administering to said subject an mTOR modulator in an amount effective for modulating mTOR activity in the subject, thereby treating or preventing retinitis pigmentosa in the subject.

9. A method for prolonging the viability of cone cells, comprising contacting the cone cells with an mTOR modulator in an amount effective for modulating mTOR activity, thereby prolonging the viability of the cone cells.

10. The methods of any one of claim 1, 8, or 9, wherein the mTOR modulator is a glucose enhancer.

11. The method of any one of claim 1, 8 or 9, wherein the mTOR modulator is selected from the group consisting of insulin, growth factors, IGF-1, IGF-2, mitogens, serum, phosphatidic acid, amino acids, leucine, and analogues or derivatives thereof.

12. The method of claim 10 or 11, wherein the mTOR modulator is insulin.

13. The method of claim 10 or 11, wherein the mTOR modulator is not insulin.

14. The method of any one of claim 1, 8 or 9, wherein the mTOR modulator stimulates mTOR phosphorylation.

15. The method of any one of claim 1, 8 or 9, wherein the mTOR modulator activates a receptor and/ or a signal transduction cascade upstream of mTOR.

16. A method for treating or preventing a retinal disorder in a subject comprising enhancing the intracellular levels of glucose in said subject, thereby treating or preventing the retinal disorder.

17. The method of claim 16, wherein the retinal disorder is retinitis pigmentosa.

18. The method of claim 16, wherein the retinal disorder is associated with decreased viability of cone cells.

19. The method of claim 16, wherein the retinal disorder is associated with decreased viability of rod cells.

20. The method of claim 16, wherein the retinal disorder is a genetic disorder.

21. The method of claim 16, wherein the retinal disorder is not diabetic retinopathy.

22. The method of claim 16, wherein the retinal disorder is not associated with blood vessel leakage and/or growth.

23. A method for treating or preventing retinitis pigmentosa in a subject comprising enhancing the intracellular levels of glucose in said subject, thereby treating or preventing retinitis pigmentosa.

24-28. (canceled)

29. A method for prolonging the viability of cone cells, comprising enhancing the intracellular levels of glucose in said cell, thereby prolonging the viability of the cone cells.

30-33. (canceled)
